(19) United States
(12) Patent Application Publication
(10) Pub. No.: US 2005/0084876 A1
(43) Pub. Date: Apr. 21, 2005

(54) Flip genes and flip proteins

(75) Inventors: Jurg Tschopp, Epalinges (CH); Martin Irmler, Cologne (DE); Kay Hofmann, Cologne (DE); Margot Thome, Dommartin (CH); Veronique Rochat, Croy (CH)

Correspondence Address: FISH & RICHARDSON PC, 225 FRANKLIN ST, BOSTON, MA, 02110, US

(73) Assignee: Apoxis SA, (formerly Apotech Research and Development Ltd.), a corporation of Switzerland

(21) Appl. No.: 10/832,218

(22) Filed: Apr. 26, 2004

Related U.S. Application Data
(63) Continuation of application No. 09/410,194, filed on Sep. 30, 1999, now abandoned.

(30) Foreign Application Priority Data
Mar. 31, 1998 (WO) PCTEP9801857
Apr. 1, 1997 (DE) 197 13 393.2

(51) Int. Cl. C12Q 1/68; C07H 21/02; C07H 21/04; A61K 48/00; C12N 5/08

(52) U.S. Cl. 435/6; 435/69.1; 435/320.1; 435/366; 536/23.1; 514/44

(57) ABSTRACT

The invention relates to nucleic acid molecules that include at least one death effector domain, expression vectors the include these nucleic acid molecules, host cells transformed with such vectors, methods for expressing and/or isolating gene products with at least one death effector domain, and purified or isolated gene products or fragments of these gene products that include at least one death effector domain.

### CROSS REFERENCE TO RELATED APPLICATIONS

This Application claims benefit from PCT Application No. PCT/EP98/01857, filed Mar. 31, 1998 and German Patent Application No. 197 13 393.2, filed Apr. 1, 1997, both of which are incorporated herein by reference in their entirety.

### FIELD OF THE INVENTION

The invention relates to DNA sequences encoding proteins or protein segments that have at least one death effector domain and inhibit cell apoptosis, and related compositions and methods.

### BACKGROUND OF THE INVENTION

Programmed cell death is known as apoptosis and occurs in embryogenesis, metamorphosis, tissue regeneration, elimination of diseased cells, and necrotization of immunologic thymocytes and T-cells. During T-cell maturation in the thymus, T-cells learn how to distinguish heterologous from autologous antigens. T-cells recognizing autologous antigens are killed off during the maturation process. The loss of these cells represents an apoptotic process.

Within the immune system apoptosis also serves as a defense strategy for eliminating potentially harmful agents. Apoptosis occurs in particular with parasites having their reproductive cycle within the cell (such as viruses, certain intracellular bacterial parasites, e.g., Mycobacterium tuberculosis, Mycobacterium leprae, Bordetella pertussis or types of Lysteria, and also protozoa, such as trypanosomas or toxoplasmas. The apoptotic processes of virus-infected cells are regulated by T-lymphocytes. This may occur in several ways. Thus, a T-lymphocyte may initiate apoptosis by releasing proapoptotic proteins (for instance perforin or granzymes), or by expressing CD95-(APO-1/Fas)-ligand (Tschopp & Hofmann, Trends in Microbiology 4, 91-94, 1996). In addition apoptosis of infected cells may in such a case also be regulated by an involuntary mechanism. The cells react to the presence of parasitizing intracellular viruses and respond to this danger by means of a cell-suicide.

In apoptosis, Fas receptors play a central role. The Fas receptor conveys an extracellular signal to the cell and, after it goes through a signal cascade, this leads to the apoptosis of the cell. Thus, for example, the Fas protein (CD95) is expressed on activated T-cells, B-cells and neutrophilic leukocytes. It is a 45 kD-protein (Itoh et al., Cell 66:232, 1991; Watanabe-Fukunaga et al., Nature 356:314, 1992). Fas-mRNA is also expressed in the thymus, the liver, the heart muscle, the lung, and/or in the ovaries of mice (Watanabe-Fukunaga et al., Journal of Immunology, 148:1274, 1992). Crosslinking of specific monoclonal antibodies against the Fas receptor leads to the induction of cell death (apoptosis) with numerous cell types (Yonehara et al., Journal of Experimental Medicine, 169:1747, 1989; Traut et al., Science, 245: 301, 1989).

There is also further evidence that in addition to the Fas receptor, other receptors, after binding of an extracellular ligand, may initiate the signal cascade leading to cell death. Thus, the membranous receptors TNFR-1 and TRAMP (wsl/DR-3/Apo-3) are involved in initiating apoptosis through binding of an extracellular ligand (Nagata, Cell 88, 355-365, 1977; Bodmer et al., Immunity 6, 79-88, 1997; Kitson et al., Nature 384, 372-375, 1996; Yu et al., Science 274, 990-992, 1996). The above receptors are also called “death receptors.” All of them belong to the tumor necrosis factor receptor family (TNF-R).

These receptors convey the “death signal” (the apoptotic signal) by means of a cytoplasmic sequence motif also called the “death domain” (DD). This death domain interacts with the adaptor molecules FADD and/or TRADD (Nagata, Cell 88, 355-365, 1997), i.e., the death domain causes an attachment of adaptor molecules. The adaptor molecule FADD is known to associate with the so-called ICE-like protease FLICE I (also called caspase-8, Mch5, or MACH) (Muzio et al., Cell 85. 817-827, 1996; Boldin, Goncharov, Goltsev, Y. V. & Wallach, Cell 85, 803-815, 1996). This association is produced via the so-called “death effector domains” (DEDs), which are present at the C-terminal of the adaptor molecule, for instance FADD, as well as at the N-terminal of the protease FLICE. The complex made up of receptor, adaptor molecule and protease is also called DISC (“Death Inducing Signaling Complex”) (Kischkel et al., EMBO J. 14, 5579-5588, 1995). The FLICE protein linked within the DISC-complex finally regulates the remaining proteolytical activities of other proteins of the ICE protease group. This proteolytical cascade finally leads to the apoptotic reaction of the cell (Muzio et al., Cell 85, 817-827, 1996; Boldin, Goncharov, Goltsev, Y. V. & Wallach, Cell 85, 803-815, 1996).

As already described, apoptosis is a defensive strategy of the immune system for killing off virus-infected cells. In their turn, viruses have developed strategies to avoid the apoptosis which would inhibit their reproductive cycle. Certain viruses are therefore equipped with genes whose genetic products block the apoptotic signal transduction mechanism (Shen, Y. & Schenk, Curr. Biology 5, 105-111, 1995). Examples are the gene products CrmA of the cowpox virus or protein p35 of the baculovirus (Shen, Y. & Schenk, Curr. Biology 5, 105-111, 1995). As expected, as inhibitor proteins these gene products above all block the effector proteases of the apoptotic signal transduction mechanism. Their inhibitory effect on the cysteine proteases, which belong to the ICE-like (or Caspase-) protein group, deserves particular mention (Henkart, Immunity 4, 195-201, 1996). But other viral genes with anti-apoptotic properties also have been identified also. These are similar to the mammalian bcl-2-gene (Shen, Y. & Schenk, Curr. Biology 5, 105-111, 1995).

Through the publications of Boldin M. P. et al. (J. Biol. Chem. 270, 7795-7798 (1995) and Chinnaiyan et al. (Chinaiyan, A. M., O'Rourke, K., Tewari, M. & Dixit, V. M., Cell 81, 505-512 (1995)), it is known that deletion mutants of the adaptor molecule FADD, then only including one death domain, or of the protease FLICE, then only including two death effector domains, exercise a dominant negative inhibitory effect on the early events of the apoptotic signal cascade.

### SUMMARY OF THE INVENTION

The present invention seeks to identify genes and their gene products that exhibit a blocking effect on cell apoptosis, and thus exercise a regulatory effect on apoptotic events.

Within the scope of the present invention, genes, nucleic acid molecules, and fragments of nucleic acid sequences, e.g., DNA, and gene products encoded by these genes, e.g., proteins or polypeptides having inhibitory characteristics toward the signal cascade, are disclosed. The invention discloses viral, human, and murine genes, various gene transcripts, and their expressed proteins and polypeptides that suppress proteolytical signal transduction, and thus block apoptosis.

The subject of the present invention therefore is a DNA sequence encoding a gene product or a gene product fragment that inhibits cell apoptosis and includes at least one death effector domain. A gene product or a gene product fragment is defined as an encoded protein or encoded protein fragment. A primary transcript, for instance mRNA, can also be called a gene product.

Within the scope of the present invention, all derivatives or alleles of the DNA sequence according to the invention are included, provided they are functionally homologous to the natural sequence. All DNA sequences currently existing in organic nature exhibiting the same functions, but showing characteristic mutations of the natural DNA sequence due to evolutionary development, qualify as alleles.

Furthermore, all derivatives of the natural DNA sequences, be they natural or artificial, are within the present invention. Artificial alterations of the natural DNA sequence can be introduced by known methods. The mutations can be introduced at a certain DNA sequence site by synthesizing the appropriate oligonucleotides including a certain mutation sequence.

In that case they are flanked by restriction sites, which promote binding of the synthesized oligonucleotides with the natural sequence. After ligation, an altered sequence with a certain amino acid, or a certain amino acid insertion, substitution or deletion is obtained.

Alternatively, oligonucleotides can be used to alter genes by means of site-specific mutagenesis. This results in changing certain codons, whereby a desired substitution, deletion, or insertion is made possible. Exemplary methods for introducing alterations into a DNA sequence have been published by, e.g., Walder et al. (Gene 42:133, 1986), Bauer et al. (Gene 37:73, 1985), Craik (Bio Techniques, January 1985, 12-19), and Smith et al. (Genetic Engineering: Principles and Methods, Plenum Press, 1981).

Variants defined as derivatives may have any desired substitutions, insertions, or deletions of a natural DNA sequence, as long as the signal cascade-inhibitory function according to the invention is present. They may exhibit conservative substitutions in which one amino acid is exchanged or another amino acid with similar physicochemical properties. Examples of conservative substitutions are the substitution of an aliphatic amino acid for another aliphatic amino acid, such as isoleucine, valine, leucine, or alanine for one another, or also a substitution of polar amino acids for other polar amino acids, for instance the substitution of lysine for arginine. Conservative substitutions also typically include substitutions within the following groups: glycine, alanine; valine, isoleucine, leucine; aspartic acid, glutamic acid, asparagine, glutamine; serine, threonine; lysine, arginine; and phenylalanine, tyrosine.

Such substitutions are well know in the art.

Within the scope of this invention, DNA sequences in which a DNA sequence according to the invention has been recombined with other DNA sequences are likewise included in the claim. This may occur through fusion at the C- or N-terminals of the DNA sequence according to the invention. Thus one might conceive of certain “reporter genes” being linked to a DNA sequence according to the invention by means of their C or N-terminals. Reporter genes could, within certain limits, reveal information regarding the quantity of the expression. On the other hand, “leader sequences” can be linked to the N- and/or C-terminal of the gene product of the DNA sequence according to the invention, but particularly at the N-terminal. Leader sequences allow the specific regulation of the import or export of the gene product into certain cell organelles or into extra-cellular space.

Segments or fragments of the gene products, e.g., peptides or polypeptides, correlating with an appropriate DNA sequence according to the invention are defined as abbreviated forms of the physiological gene product. These abbreviated forms may be abbreviated either at the N- or the C-terminals of the natural sequence by means of a process known per se. However, abbreviated forms lacking an amino acid sequence fragment within their internal sequence are also part of the invention. Likewise, various internal amino acid sequence segments can also be removed from the natural amino acid sequences. The pertinent DNA sequences, in which degeneration of the genetic code for one amino acid sequence always results in a multitude of DNA sequences in accordance with the invention, in all of the above cases, correspond to the inventive idea.

The condition for having a DNA sequence, be it a derivative of the natural DNA sequence or an allele of the natural DNA sequence, or a segment of the natural DNA sequence of any composition within the invention, is that the DNA sequence should encode a gene product or a fragment of a gene product that inhibits cell apoptosis.

Within the scope of this invention, DNA sequences inhibiting cell apoptosis under physiological conditions by means of at least one death effector domain are being disclosed for the first time.

### BRIEF DESCRIPTION OF THE DESCRIPTION OF FIGURES

FIGS. 1a-1 and 1a-2 are two sheets of a search profile based on FADD, FLICE and Mch 4.

FIGS. 1b-1 and 1b-2 are two sheets of a search profile based on FADD, FLICE, MCh 4, and viral inhibitors.

FIGS. 2a to 2d are four sheets of a representation of amino acid sequences of EHV-2 (E8), HHV-8 (71), HVS (71), BHV-4 (ORF), MCV (1591), MCV (16L) FLICE, Mch4, FADD, and PEA-15.

FIGS. 3a to 3h are nine sheets of a representation of amino acid sequences of FLIP-H5, FLIP-MM, wFLIP-EHV2, FLICE-HS, and Mch4-HS.

FIG. 4a is a representation of the nucleotide and deduced amino acid sequence of the human c-FLIP_{S} gene.

FIGS. 4b-1 and 4b-2 are two sheets of a representation of the nucleotide and deduced amino acid sequence of the human c-FLIP_{L} gene.

FIGS. 4c-1 to 4c-3 are three sheets of a representation of the nucleotide and deduced amino acid sequence” of the murine c-FLIP_{L} gene.

FIGS. 5a and 5b are Western blot results of cotransfection experiments in 239T cells using viral FLIP genes.

FIGS. 6a and 6b are two representations of gels showing the attachment of viral FLIP proteins to the CD95 receptor in human Raji B-cell clones.

FIGS. 7a and 7b are two representations of electrophoresis gels showing the association of viral proteins with the stimulated CD95 death receptor.

FIGS. 8a, 8b, and 8c are graphs showing that eukaryotic cells expressing viral FLIP proteins are more resistant to apoptosis than control cells.

FIGS. 9a, 9b, and 9c are graphs showing the shielding effect of viral FLIP_{S} against induced apoptosis.

FIG. 10 is a graph showing cell proliferation of various cell types as evidence of resistance to apoptosis induced by TRAIL, through the expression of viral FLIP proteins.

FIGS. 11a and 11b are a gel and a graph showing the correlation of the expression of a viral FLIP protein in the course of viral infection of a host cell with the shielding effect against induced apoptosis.

FIGS. 12a and 12b are gels showing binding of human FLIP (long and short forms) to FADD.

FIG. 13 is a representation of a Western blot gel showing binding of human FLIP proteins to FLICE.

FIGS. 14a, 14b, and 14c are graphs and corresponding gels showing resistance to induced apoptosis in Jurkat T-cells expressing human FLIP proteins.

FIG. 15 is a graph showing that human Jurkat T-cells acquire resistance to apoptosis induced by TRAIL through expression of FLIP proteins.

FIG. 16 is a representation of the amino acid sequence of ORF71 of human herpesvirus 8 (HHV8).

FIG. 17 is a representation of the ORF of bovine herpes virus 4 (BHV4).

### DETAILED DESCRIPTION

Death effector domains have so far been known only as protein-binding domains with the function of transmitting the extracellular signal for initiating apoptosis. In this case the death effector domain served to bind caspase proteins to adaptor molecules to initiate apoptotic events. Thus, the death effector domain has an activation effect on the transmission of the signal that leads to the apoptosis of the cell. This has also up to now been the functional definition of the death effector domain. It serves as a signal-transmitting, linking domain within the scope of the signal cascade required to activate apoptosis (Nagata, Cell, 88, 355-365, 1997).

The nucleic acid molecules according to the invention, on the other hand, code for proteins or protein fragments which do not serve to transmit the signal for initiating apoptosis, but instead, block the signal cascade that initiates the apoptotic events. Therefore, all DNA sequences having an inhibitory effect on cells that have already been prepared for apoptosis by an external signal are the subject of this invention, as long as they encode at least one death effector domain. Any derivatives or alleles, by no means limited to those enumerated in the present examples, are included in the subject of the invention if they likewise interrupt the apoptotic signal cascade. This makes them functionally analogous to the gene product of the natural nucleic acid molecules according to the invention. With respect to the regulation of the signal cascade, the nucleic acid sequences according to the invention, together with their gene products, thus perform functions exactly opposite those of the known proteins with death effector domains, such as FLICE or Mch4.

Therefore, quite surprisingly, the disclosure of the present invention has revealed that there are DNA sequences encoding proteins with death effector domains that may prevent cell apoptosis in the most varied life forms, such as viruses and mammals.

Thus, the expression of a nucleic acid sequence according to the invention may save 70% and possibly more than 90% of the cells from apoptosis after addition of an apoptosis-stimulating agent, for instance by addition of the CD95 ligand.

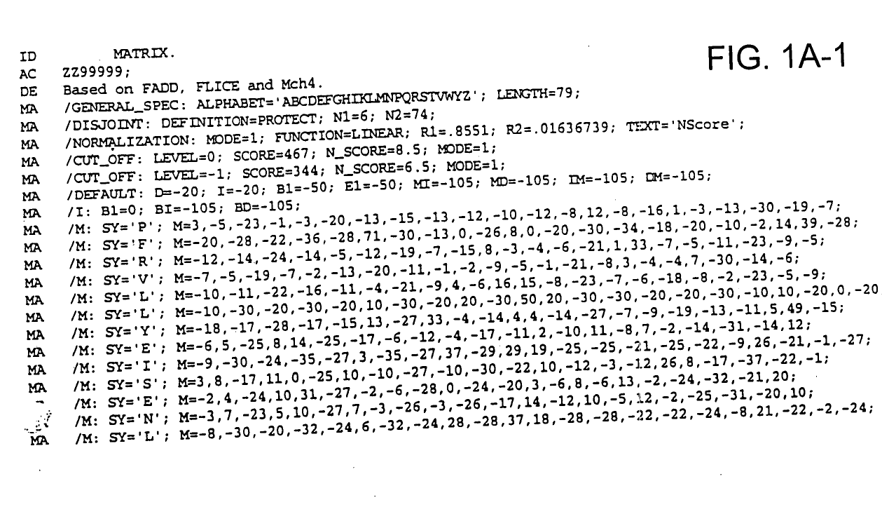

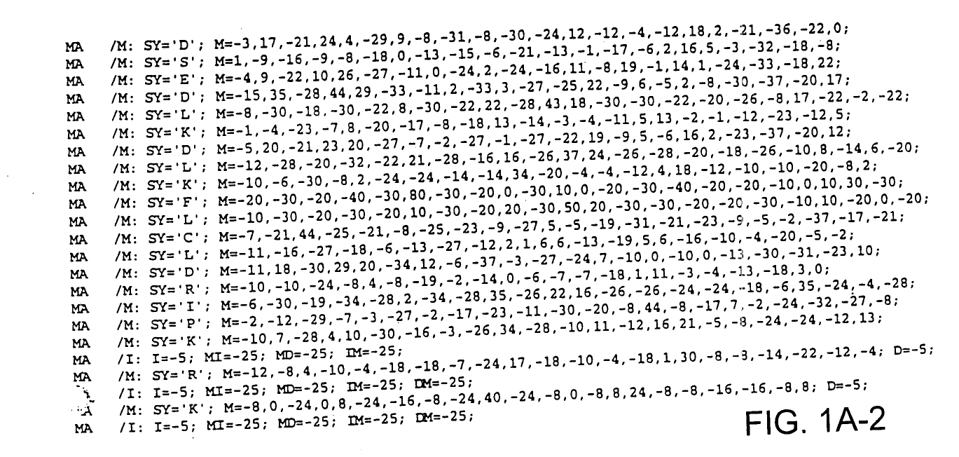

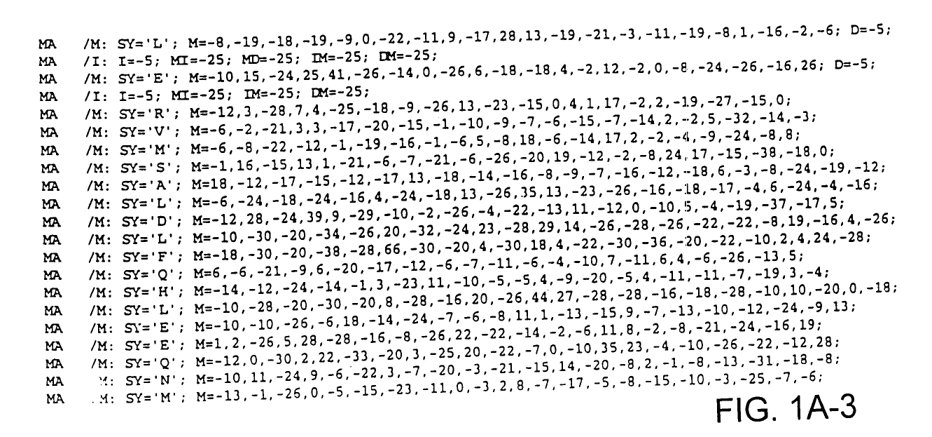

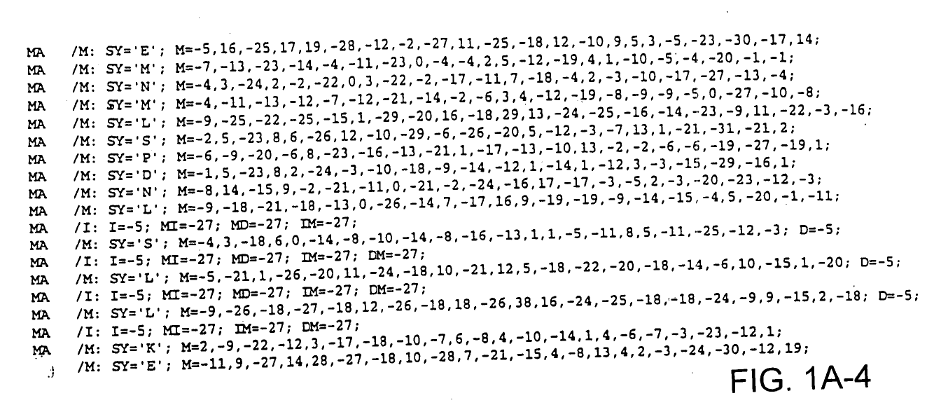

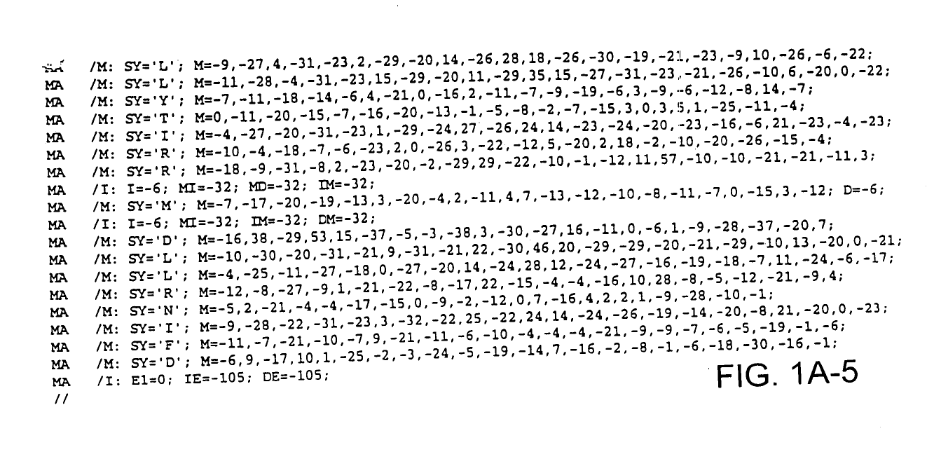

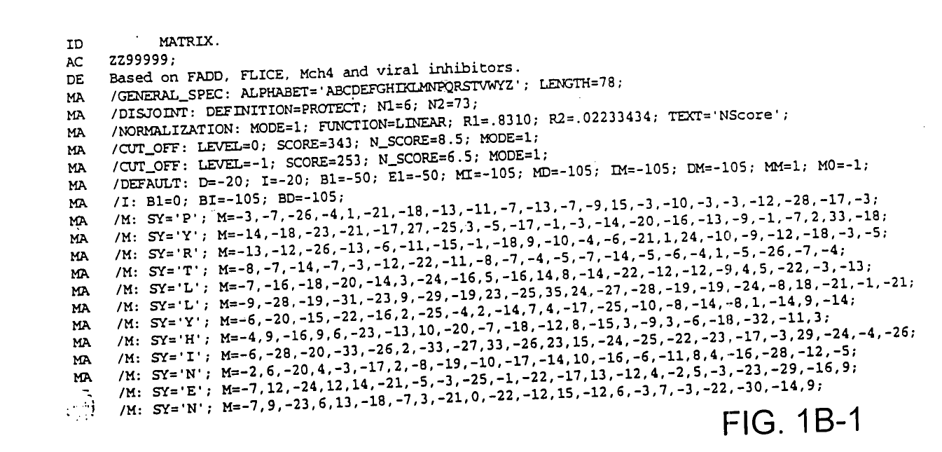

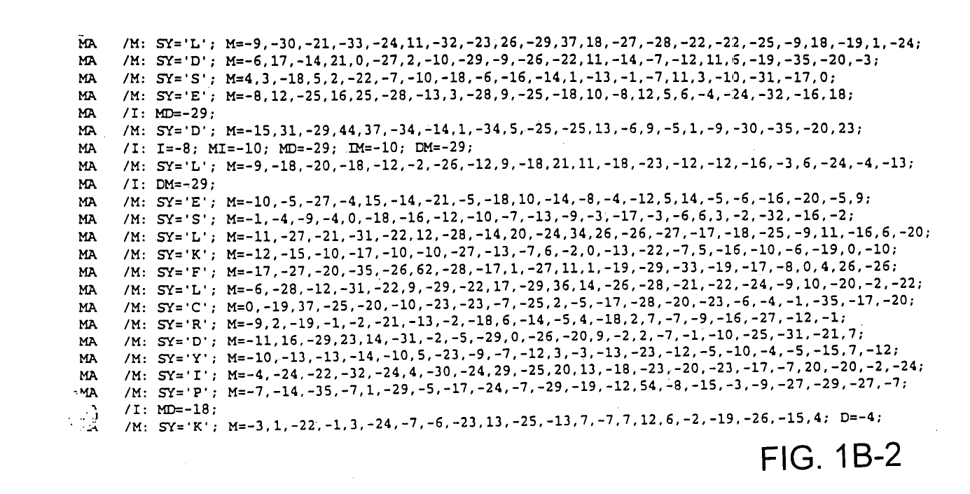

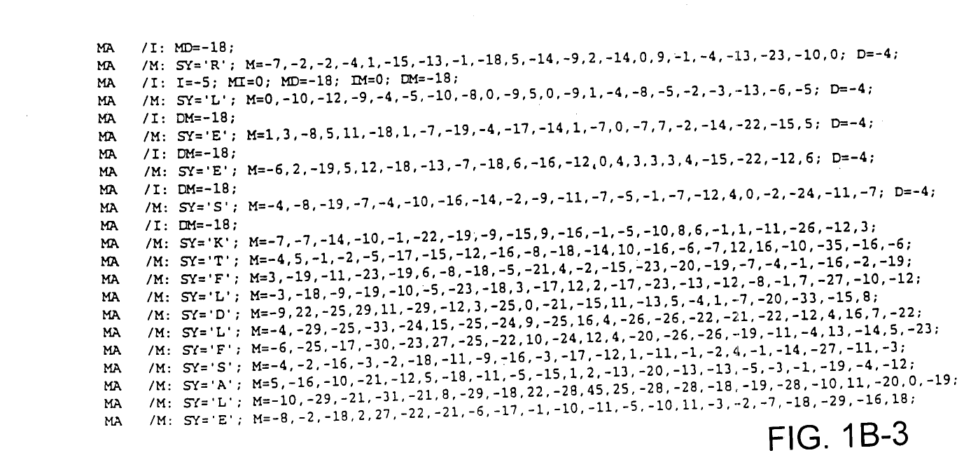

In a preferred embodiment of the DNA sequence according to the invention, a DNA sequence is claimed which exhibits a significance level of p<10⁻² in a comparison of the sequence of the death effector domain with a search profile according to FIG. 1a or a search profile according to FIG. 1b. Such a search profile is established when related sequences with homologous functions are to be identified within the scope of a data base search.

Indeed a multitude of protein sequence currently are stored in data bases for example, the data bases of SWISSPROT, established by EMBO (Eur. Mol. Biol. Org.), or the database “GenBank” (GenBank data base, s. Benson et al. (Nucleic Acids Research 25: 1-6 (1997)), each of them with stored DNA sequences and/or amino acid sequences of proteins. The physiologically closely related function for most of these DNA sequences and/or amino acid sequences is not known. By using sequencing homologs, and based on sequences of known functions, an attempt is being made to identify similar sequences within the data base possibly having the same or at least physiologically closely related functions. The success of such a data base search on the one hand depends on the success of such a data base search, but on the other hand, the search profile, which is being created in accordance with the sequence or sequences of known functions, is of key importance. In accordance with the invention, two search profiles were designed for proteins exhibiting so-called death effector domains as described above. The amino acid sequences for each of the proteins FADD, FLICE and Mch4 (also called caspase-10), coded by the appropriate DNA sequences, served as a basis for the profile design for the present invention (Fernandes-Alnemri, T., et al., Proc. Natl. Acad. Sci. USA 93, 7464-7469 (1996)) on the one hand. This profile can be seen in FIG. 1A.

FIG. 1b, on the other hand, shows a search profile derived from a generalized search profile on the basis of six amino acid sequences coded by viral DNA sequences according to the invention (see below). These DNA sequences according to the invention represent gene, which are called FLIP genes herein, except if derived from viruses, in which case they are called vFLIP genes, which encode vFLIP proteins. The profiles are hereby aimed at the amino acid sequences of the known death effector domains. According to the profile, newly identified proteins or DNA sequences shall include at least one death effector domain. The data base search was carried out with the algorithm by Bucher et al. (Bucher, P. Karplus, K., Moeri, N. & Hofmann, K. A., Computer Chem. 20, 3-24 (1996)). The present amino acid sequences and the DNA sequences on which they are based were identified in the database “GenBank.” However all commercial or non-commercial data bases with amino acid sequence entries may come under consideration for homology searches. A search in DNA data bases with a DNA search profile for death effector domains is another possibility. The invention is not limited by the current status of the entries, that is, by the number of protein sequences entered by the filing date for the patent. All future data base entries with their pertinent DNA sequences according to the invention and their corresponding protein sequences are included in this preferred embodiment of the invention, provided they meet the design criteria of the profiles in FIG. 1A or 1B.

In accordance with the invention under a preferred embodiment, a significance level of p<10⁻² must result when the sequence of the death effector domain is compared with a search profile according to FIG. 1A or a search profile according to FIG. 1B. In order to calculate the statistical significance of the hits identified in the data base according to the search profile, the pertinent p-values reveal statistical significance values resulting from a conversion of normalized profile scores (Nscores). The programs pfscan and pfsearch (both included in the program package pftools) in their output file yield so-called “normalized scores” (Nscores) as a comparison standard for the significance of a profile/sequence. Pftools is a freely available program package for generalized profile applications. It has been described in the publication by Bairoch et al. (Nucleic acids Research 25: 217-221 (1997). It is available on the Internet under ftp://ulrec3.unil.ch/rub/pftools or by inquiring with Dr. Philippe Buchner, Swiss Institute of Exp. Cancer Research, H-1066 Epalinges, Switzerland. The program pfsearch is used for searching a sequence data base using a profile, while the program pfscan is a tool for searching a profile data base by means of a sequence. The normalized scores are calculated from raw scores by applying scaling parameters. Some of the scaling parameters are derived through profile construction based on a search in a randomized data base, whereby the parameters become part of the profile. The randomized data base, which may, for instance, be based on the SWISSPROT data base, is then created by means of so-called “sectional shuffling” with a window latitude of 20 residuals (Pearson and Lipman, Proceedings of National Academy of Science, USA, 85, 244-2448, 1988). Further information about the algorithm can also be found in the publications by Bucher et al. (Computer Chemistry 20, 3-24, 1996) and Hoffmann et al. (TIBS 20, 347-349, 1995). Nscores may be interpreted as a base 10 logarithm of the number of amino acid residues in a randomized data base, in which one would accidentally find exactly one protein of the desired quality. One may, for instance, state that an Nscore of 9.0 may be expected to occur exactly one time by accident in a randomized date base of 10⁹ residues. Since the protein data base currently contains around 58,000,000 residues, which corresponds to a magnitude of 10^{7.76}, a protein with an Nscore of 7.76 could, according to the statistical average, be expected to occur once. Higher Nscores signify better protein/profile hits than could be expected in a data base of the current size by pure accident. In practice an Nscore of 8.5 to 8.7 has established itself as a significance limit. The probability p of finding such a hit in the protein data base by accident is about 0.1. This is based on the following calculation:
p=10^{-(Nscore-log(data base size])}.
This equation holds for Nscores>8.5.

In sum, it may be said that the probability of error has hereby been defined as the probability that a hit of the desired quality will be accidental and does not have any evolutionary relationship to a cause. Further information about the search method in a data base by means of a search profile may be found at the following web sites: http://u1-rec3.unil.ch/profile/profile/html, and http://u1rec3.unil.ch/profile/scoredoc.html.

A special embodiment is represented by the coding of the DNA sequences for proteins according to the invention, that have two death effector domains. The presence of two death effector domains permits a strong link with the protein to be bound, for instance to the adaptor molecule FADD. While the two death effector domains of the DNA sequence according to the invention show distinct similarities, they preferably are not identical. Variations in the amino acid sequence of the two death effector domains result in a specificity of each domain that is functionally significant. Therefore the linking behavior of the proteins coded by the DNA sequences according to the invention with proteins of the apoptosis signal transduction path, may be optimally regulated. Given the cellular concentration of the coded protein according to the invention, the intensity of the apoptotic signal may be regulated through the binding constant. The scope of this invention, however, in addition lays claim to artificially recombinant DNA sequences, which code for proteins with two identical death effector domains. The recombination of certain DNA sequences according to the invention (for instance, of DED1 from HHV-8 and DED2 from MCV (159L) or duplication of DED1 from HHV-8 or similar combinations) allows certain differentiated inhibitory effects on the apoptotic reaction to be achieved.

In a particular embodiment of the present invention the proteins (or gene products) encoded by the DNA sequences according to the invention bind to a protein of the apoptosis signal transduction path. This provides a way for the gene products coded by the DNA sequence according to the invention to intervene in the apoptosis signal transduction path. Proteins including death effector domains make particularly good target proteins for the linkage. Within the signal transduction path these target proteins can either function as adaptor proteins, as does FADD, or as proteases, especially as caspase-like proteases. In this manner, signal transmission from one protein of the apoptosis signal transduction mechanism to the following elements in the signal cascade may be blocked. In that case the apoptosis signal triggered outside the cell that has been transmitted via the receptor is dead-ended. Further signal transmission to the proteolytic members of the signal cascade therefore is no longer possible. Apoptosis of the cell is prevented.

The specific binding of gene products of the DNA sequences according to the invention by means of one or several death effector domains of proteins in the apoptosis signal transduction path enjoys particular preference.

Particular preference is given to those DNA sequences whose gene product matches the search profiles shown and who at the same time, in their earliest intracellular stage of apoptosis signal transmission, already produce a blockage of the extracellular apoptosis signal. This occurs through gene products which bind directly or indirectly to the cytoplasmic segment of a membranous, cellular receptor of the apoptosis signal transduction path.

This is the case above all with the receptors for the tumor necrosis factor type. Worth mentioning are the receptors TRAMP, CD95, and TNFR-1, and also the receptor for the death-inducing ligand TRAIL (Wiley et al., Immunity 3, 673-682 (1995)).

In addition, those DNA sequences are particularly preferred which encode gene products that bind to soluble intracellular proteins of the apoptosis signal transduction path. An interaction between the gene product of the DNA sequence according to the invention with the protein FADD may be mentioned as an example. If this interaction takes place via the death effector domains involved, then an interaction between the adapter molecules, such as FADD or TRADD, and the cytoplasmic segment of the receptor still can occur. However, signal transmission from the adapter molecule to the proteins of the signal cascade functioning proteolytically will be blocked. This means that interactions between the death domains of the cytoplasmic segment of the apoptosis receptor and the adapter molecule still are possible, but that the subsequent signal transmission step, for instance from FADD to FLICE, has been blocked.

To sum up, a preferred embodiment of the DNA sequences according to the invention encode a gene product that attaches to an adaptor molecule in such a manner that a complex made up of the receptor in the form of its cytoplasmic segment, of the adaptor molecule, and the gene product of the DNA sequence is created, and thus inhibition of the apoptotic signal cascade is assured.

As the signal cascade of apoptosis progresses, the DISC-complex described above is formed. The DISC-complex is composed of several elements which are deposited on the CD95 protein. The elements are named CAP1 to CAP6, whereby CAP1 represents a FADD protein, CAP2 a pro-FLICE protein, CAP3 an until recently unknown protein with the N-terminus of the FLICE terminus, CAP4 a pro-FLICE protein, and CAP5 or CAP6, FLICE domains already split off, which may serve as indicators during an activation of FLICE protein within the DISC complex. In case of an agonistic stimulus to the CD95 receptor, these components are deposited together with the CD95 protein. In the presence of a gene product of the DNA sequence according to the invention, the DISC complex is not fully completed, although CAP1, as expected, deposits on the CD95 protein, because at the same time the presence of CAP4 and CAP3 (each of which is a FLICE or FLICE-like component) has been severely reduced. Beyond that, the components CAP5 and CAP6 cannot be detected.

Within a special embodiment therefore, the deposit and activation of the FLICE protein is nearly blocked by the gene product of a DNA sequence according to the invention. Therefore no components occurring later in the signal cascade and functioning proteolytically can be activated. The apoptosis signal is therefore being interrupted. Just as with the CD95 receptor, so too with the TRAMP receptor, the FLICE protein is activated by association with the adaptor proteins TRADD and FADD. Here too, inhibition of exogenously stimulated cell death by means of a co-expression of gene products of the DNA sequences according to the invention will occur. These gene products of the DNA sequences according to the invention may also block apoptosis signal transmission if the protein TRAIL, likewise a member of the TNF-ligand class, is added exogenously to the cells as an apoptosis stimulant. In all of the cases mentioned there is, however, a dose-response correlation between blockage of the apoptosis signal and the concentration of the gene product of the DNA sequences in accordance with the invention.

In a particularly preferred embodiment, the gene product of the DNA sequences according to the invention completely inhibits cell apoptosis by preventing signal transmission to the proteolytical proteins in the cascade.

In another embodiment, the DNA sequences according to the invention occur in viral, prokaryotic, or eukaryotic DNA-hereditary informational molecules. The DNA sequences according to the invention therefore occur in all forms of life.

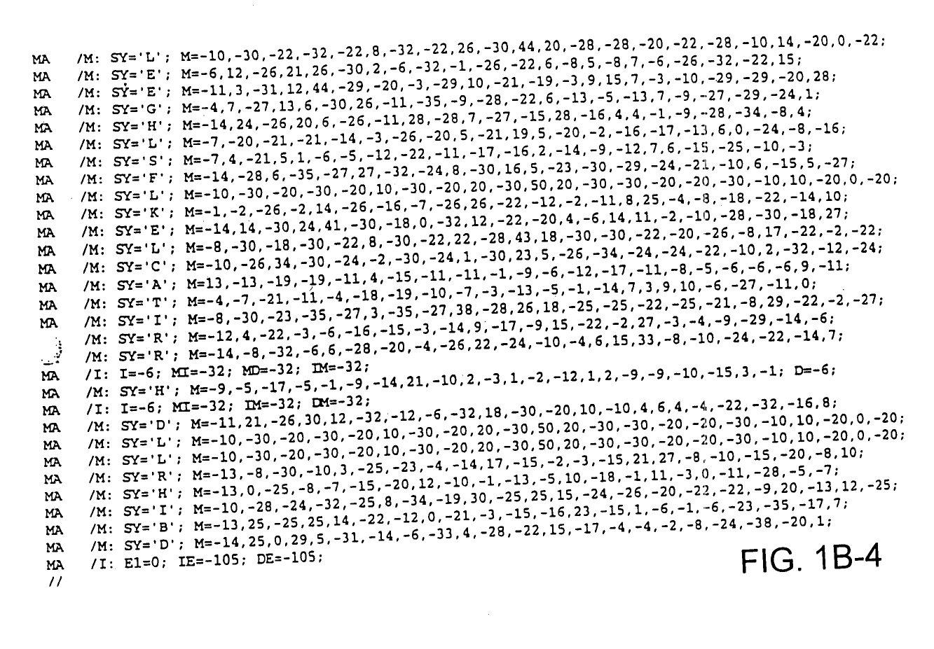

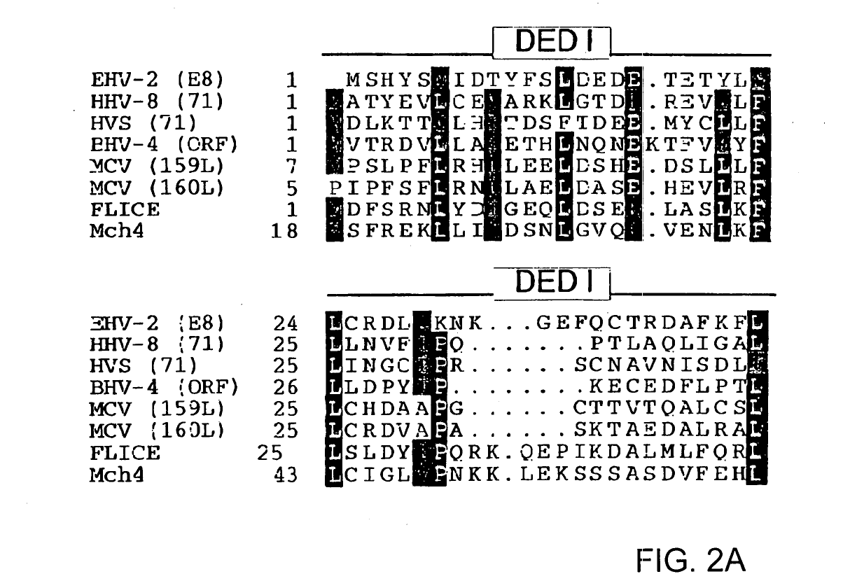

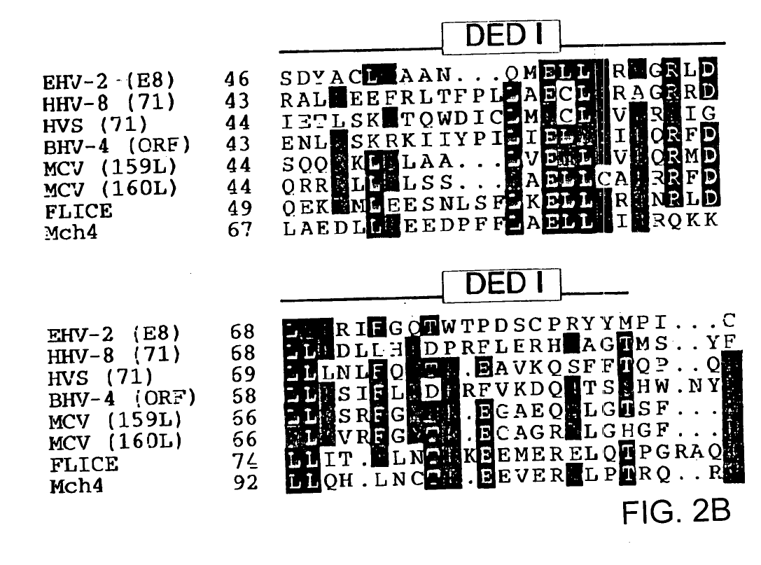

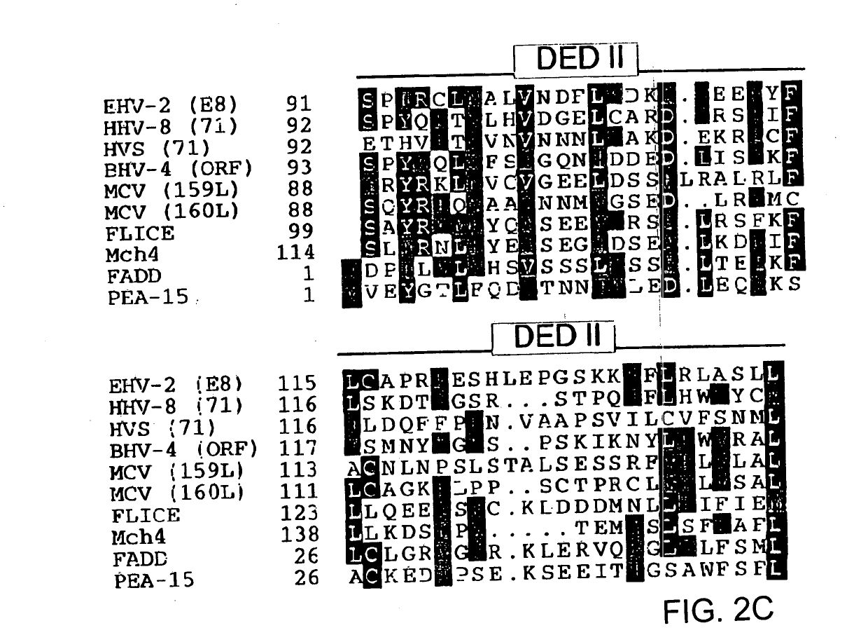

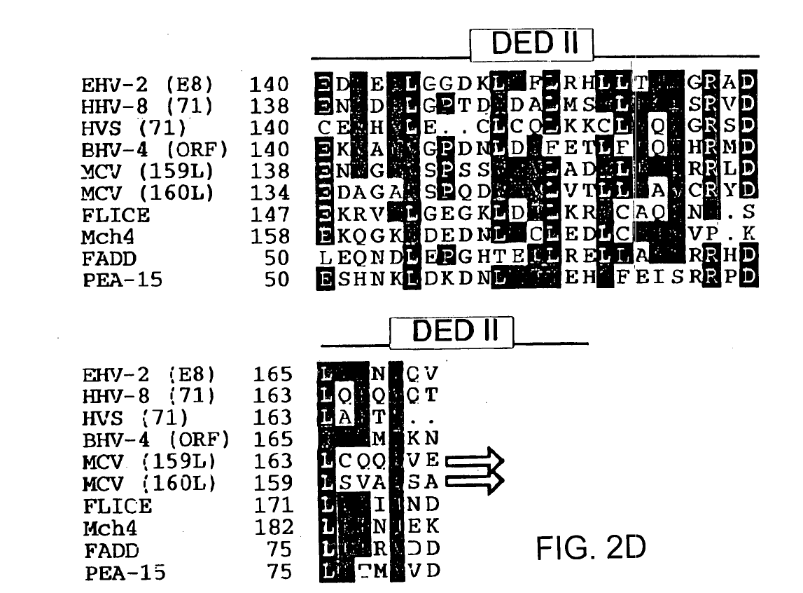

DNA sequences leading to the expression of gene products, with at least one death effector domain, and inhibiting cell apoptosis, mainly occur in viruses. Here the use of the search profile according to FIG. 1a or FIG. 1b has resulted in identification of several proteins, whose sequence is homologous to the known cellular apoptosis signal proteins FLICE and Mch4. DNA sequences according to the invention occur, for example, in the molluscum contagiosum virus (MCV). Here in particular human MCV deserves mention as a carrier of the inventive sequences. With this virus a DNA sequence according to the invention occurs in ORF 159L or in ORF 160L. These ORFs code for proteins that may inhibit cell apoptosis. Amino acid sequences of DNA sequences according to the invention have been entered in the data base GenBank with an access code of U60315 (for the ORF 159L or for the ORF 160L of the MCV). In addition, N-terminal amino acid sequences of the two ORFs are shown in FIGS. 2a-d. This site has two death effector domains. Both amino acid sequences have a C-terminal extension indicted by arrows in FIGS. 2a-d. The extensions are either 66 amino acids long (ORF 259L), or 102 amino acids long (ORF 160L).

In a particularly preferred embodiment the DNA sequences according to the invention occur with herpes viruses, particularly with γ herpes viruses. These herpes viruses are ones with double-stranded DNA, whose morphogenesis occurs in the nucleus of the host cell.

Another DNA sequence encodes a gene product including an amino acid sequence set forth in GenBank accession code U20824 (E8), where E8 indicates the ORF. This amino acid sequence is derived from the virus EHV-2. With this sequence it is remarkable that in four areas of the domain DED I no agreement exists with the pertinent sequences otherwise listed among those in FIGS. 2a-d. In these positions the EHV-2 sequence alone shows deviations.

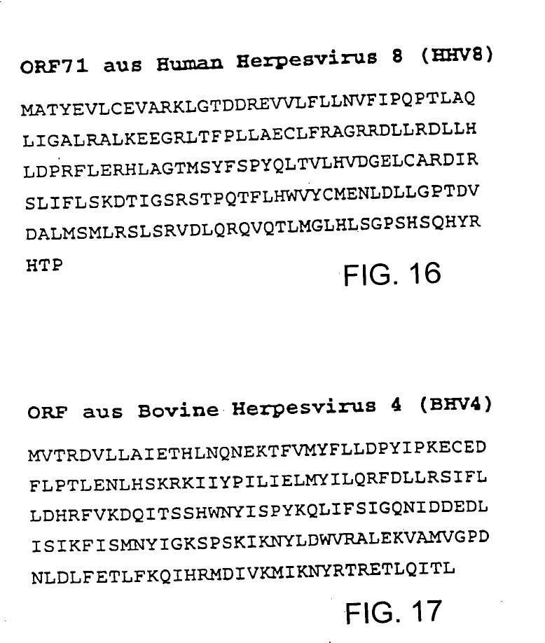

Another DNA sequence encodes a gene product including an amino acid sequence set forth in FIG. 16. It represents an amino acid sequence of the virus HHV-8. This sequence is in ORF 71. A DNA sequence according to the invention on which it is based has been listed in the GenBank under access code (U90534 (71)).

In addition, another DNA sequence encodes a gene product including an amino acid sequence set forth the GenBank access code X64346 (ORF 71). This sequence is present in the HSV virus.

Finally, another DNA sequence encodes a gene product that includes an amino acid sequence set forth in FIG. 17. This viral sequence (BHV-4) has a GenBank data entry for a DNA sequence in accordance with the invention under Z46385.

As defined by this invention, these genes or gene products are called vFLIP genes or vFLIP proteins (abbreviation for viral FLICE Inhibition Proteins).

Here are found two sequence motifs with a highly significant homology for the death effector domains. The significance level p for the search profile according to FIG. 1a or 1b according to the invention is smaller than 10⁻². All of them have two death effector domains each (DED I and DED II). These were determined via the search profile shown in FIG. 1a using the algorithm by Buchner et al. (see above). They thus exhibit the characteristic sequence motifs of a death effector domain.

In order for viral FLIP proteins to function, at least one death effector domain, but preferably two domains of this type, are necessary. With these five viruses (EHV-2, HHV-8, HVS, BHV-4, and MCV) the two death effector domains are linked by a sequence of at least 16 amino acids. Within this linkage sequence, only a weak homology between the five viruses mentioned exists. The linkage sequence preferably has a length of 16 to 19 amino acids. The DNA sequences according to the invention may encode gene products that include at least one or both of these death effector domain amino acid sequence segments.

In an embodiment of the present invention, the DNA sequence according to the invention is present within the hereditary information of mammalian cells. In this connection all imaginable mammalian cells are included. The invention relates particularly to human DNA sequences of the type defined by the invention.

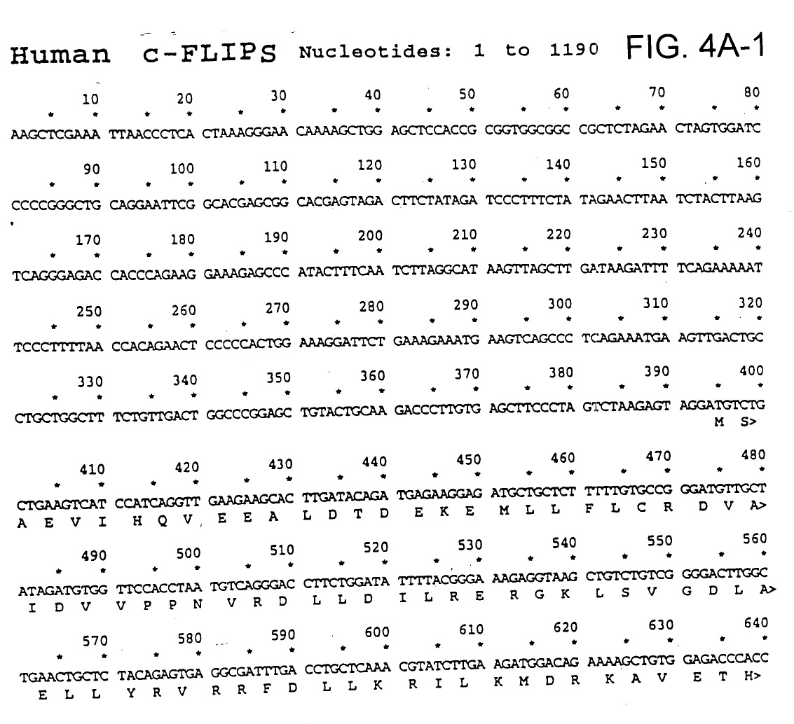

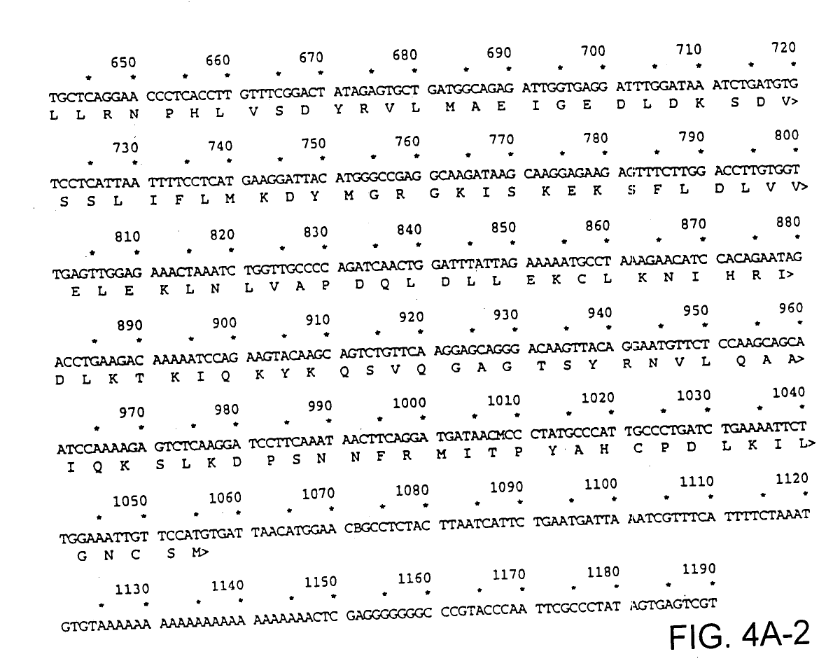

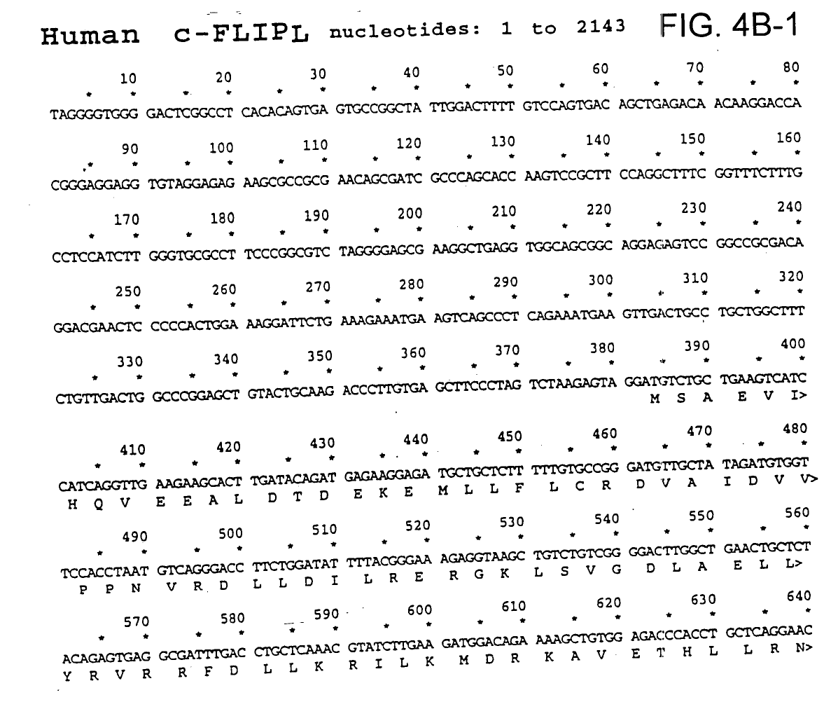

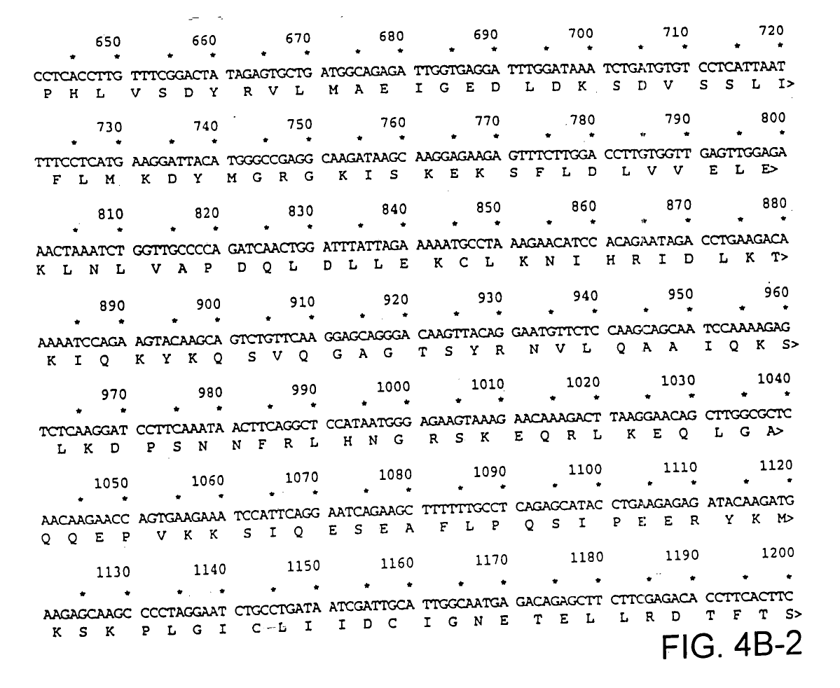

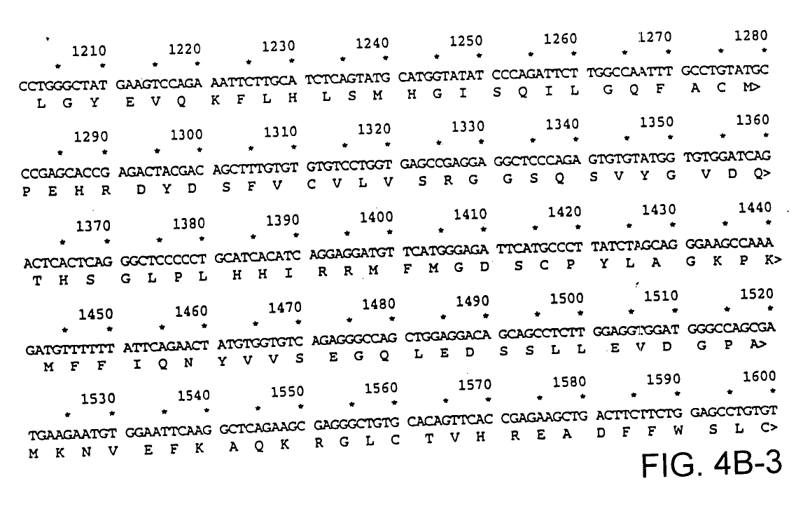

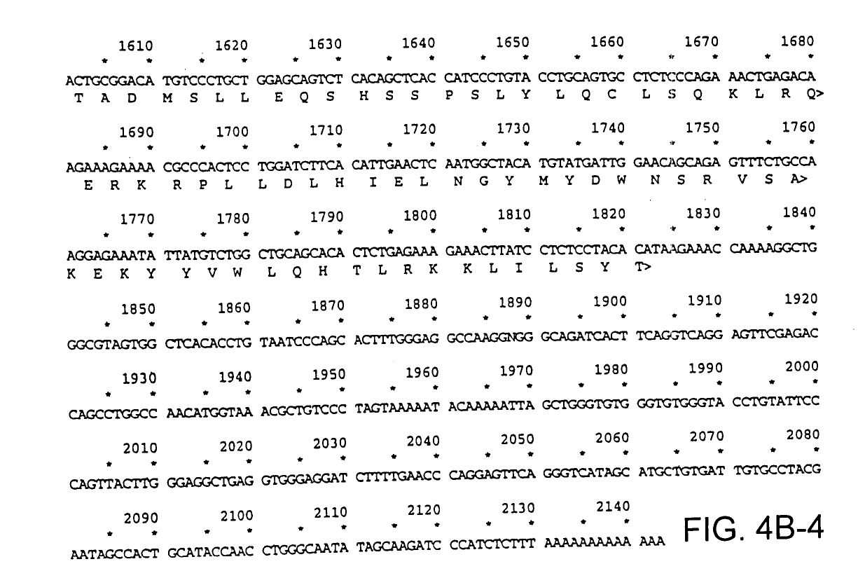

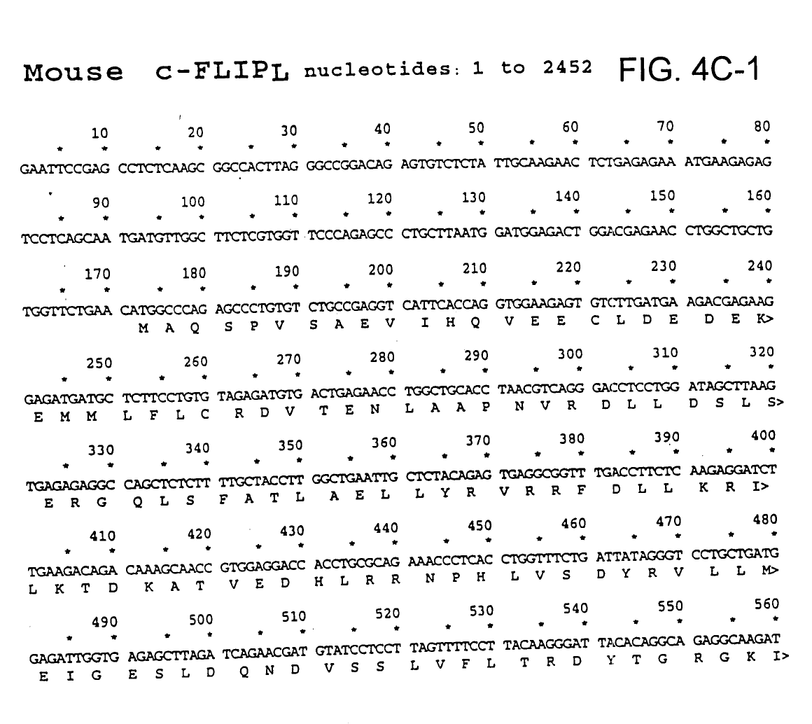

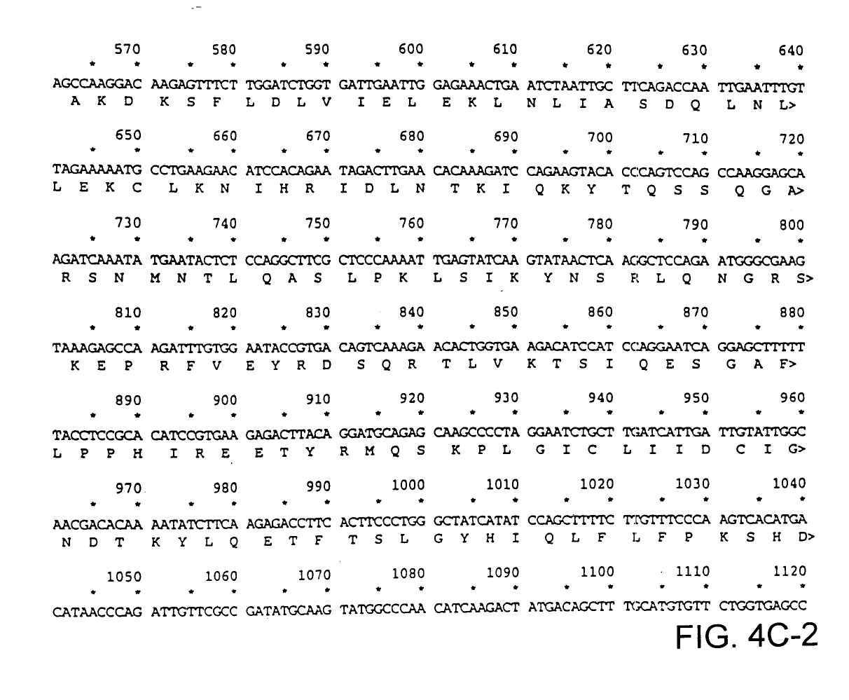

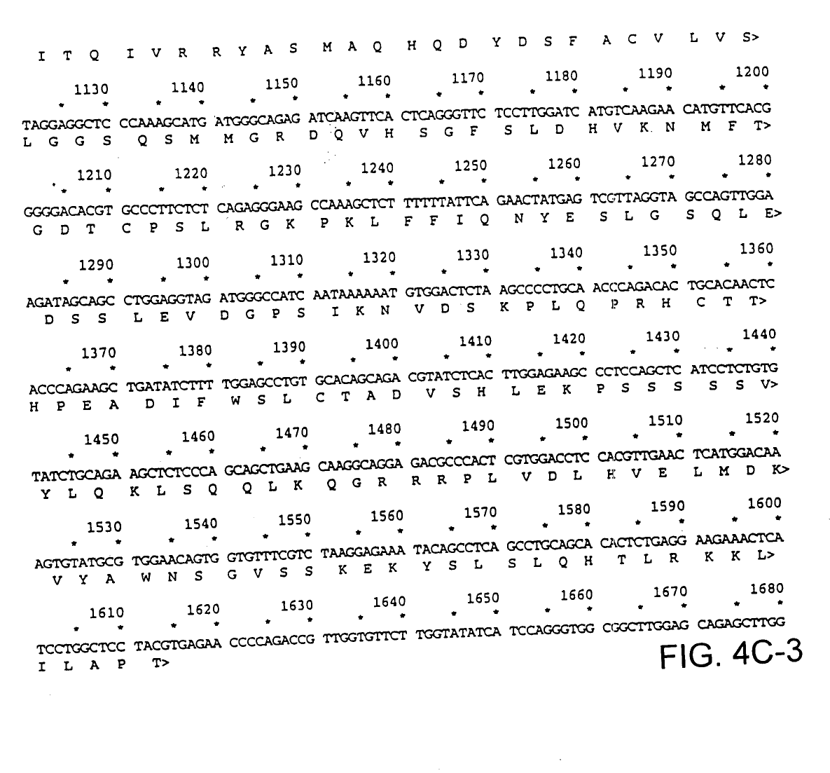

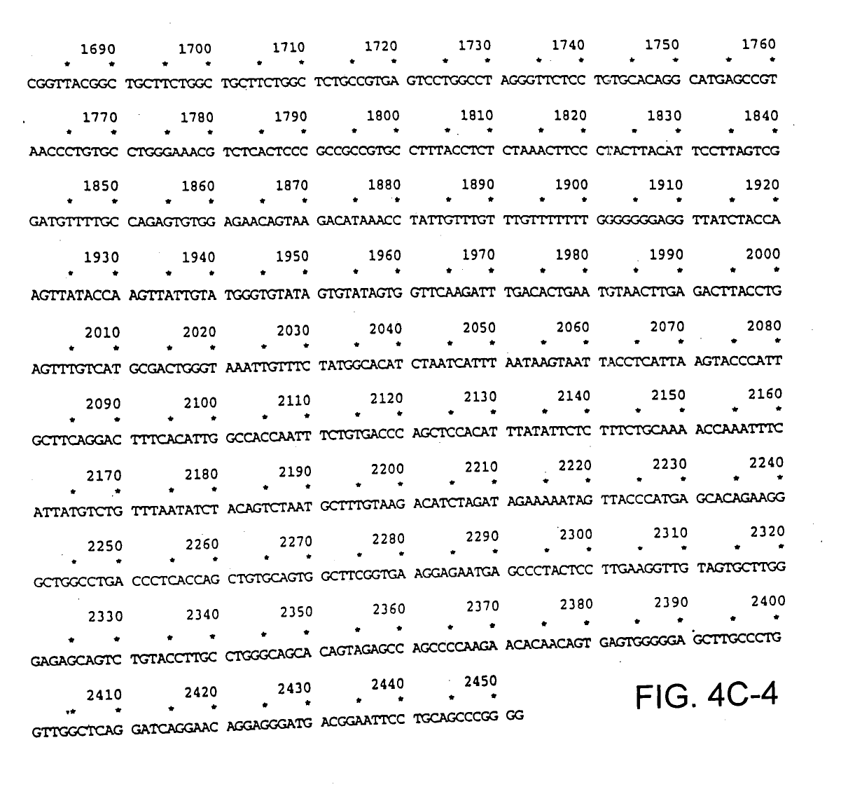

A DNA sequence is included in the subject of the invention particularly if its gene product has or includes an amino acid sequence, as is shown in one of FIGS. 4a, 4b, or 4c. FIGS. 4a and 4b show the amino acid sequences and the DNA sequences of human proteins pertaining to them that are homologous with the viral vFLIP proteins. The sequences were determined by screening cDNA libraries of activated human peripheral hemolymphocytes (PBL). During this procedure several clones encoding a protein closely related to the viral FLIP protein were identified. This protein has two death effector domains (DED), which allow only a short C-terminal sequence to connect. In the following this human protein will be known as FLIP_{S}. A human FLIP_{S} DNA sequence in accordance with the invention is shown in FIG. 4a. It includes 1190 nucleotides.

A longer version of a human FLIP-DNA sequence is illustrated in FIG. 4b. This kind of DNA sequence according to the invention, for coding a protein, called FLIP_{L} herein, and also isolated from activated human peripheral hemolymphocytes, includes 2143 nucleotides. FLIP_{L} and FLIP_{S} are alternative splice variants of the DNA sequence according to the invention. According to the invention, all splice variants exhibiting the characteristics of the invention, specifically that of encoding a gene product with at least one death effector domain and at the same time, inhibiting cell apoptosis, are included in the present invention. The invention demonstrates that three different RNA species exist in the form of transcripts of the DNA sequence according to the invention, that is, a DNA sequence of at least one and preferably two death effector domains with apoptosis-inhibiting activity. The shortest RNA transcript (1.1-1.3 kB) shows the highest expression in muscle tissue and in the peripheral hemolymphocytes. This shortest transcript does not include a caspase-homologous domain, as is the case with the longer version. In addition, two longer transcripts were detected (2.2 kB and 3.8 kB respectively) both of which exhibit a caspase-homologous domain. Compared to the number of the shorter FLIP_{S} transcripts, the number of the longer FLIP_{L} transcripts synthesized in the cell is small. They are mainly limited to muscle and lymphatic tissue, particularly to the spleen and small intestine, or to peripheral hemolymphocytes. Small numbers of transcripts may however also be detected in other tissues.

FIG. 4c additionally shows a murine DNA sequence, a long version with a caspase-homologous domain. Its amino acid sequence is also revealed in FIG. 4c. The DNA sequence of this cellular FLIP protein has 2452 nucleotides. The murine gene was identified within a cDNA library of murine cardiac cells using human cDNA samples.

The present invention includes DNA sequences made up of cellular DNA of eukaryotic FLIP_{L} sequences that, besides at least one death effector domain, also exhibit a caspase-homologous domain. FLIP_{S} sequences, on the other hand, have only at least one death effector domain.

An embodiment of the present invention claims a DNA sequence whose gene product has two death effector domains and one caspase-homologous domain, whereby the caspase-homologous domain is not functional. A Caspase-homologous domain is defined as a domain of the proteases of the caspase type (i.e. the cysteine-protease type), whereby this homologous domain equips the caspases for their proteolytical activity. A non-functional caspase-homologous domain is characterized by no longer exercising any cysteine-proteolytical activity. This type of functional loss may be based on a sequence-specific mutation. With the particularly preferred DNA sequences for gene products of the long cellular FLIP proteins, the cysteine residue within the active center critical to proteolytical activity, which for instance equips FLICE protein for its proteolytical activity, mutates.

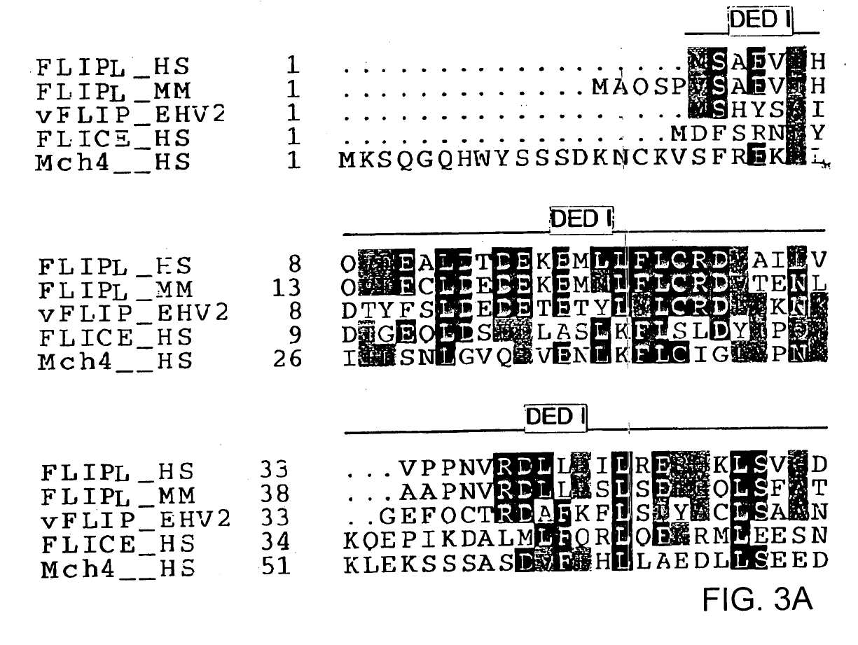

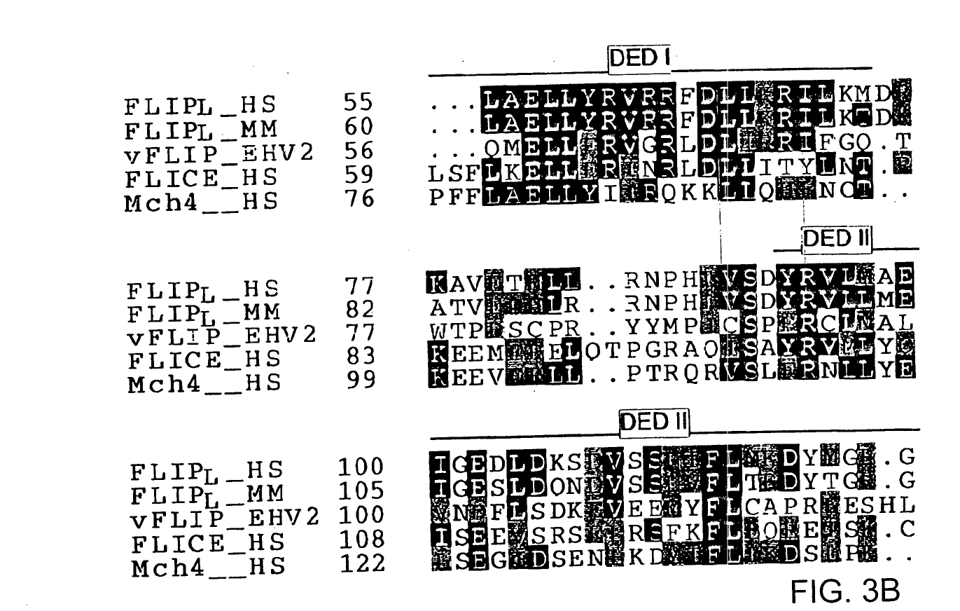

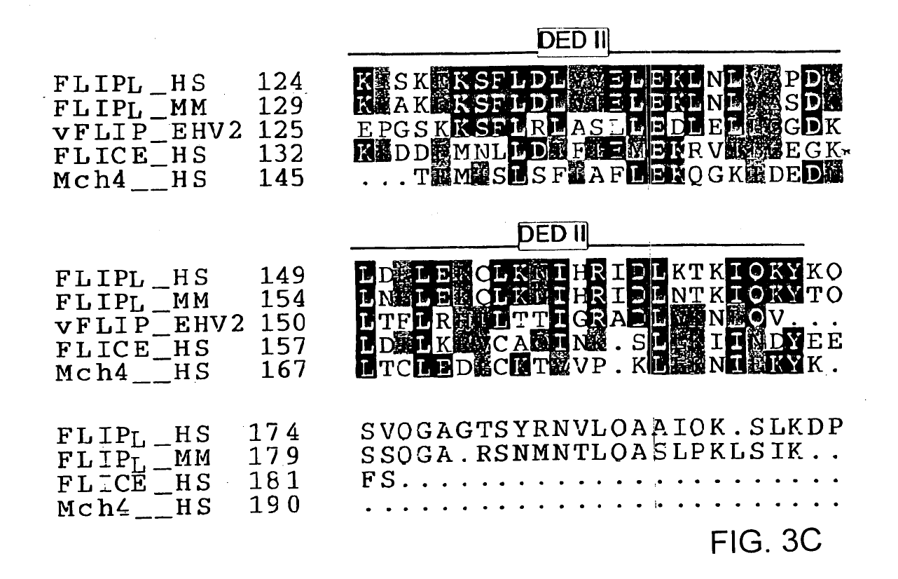

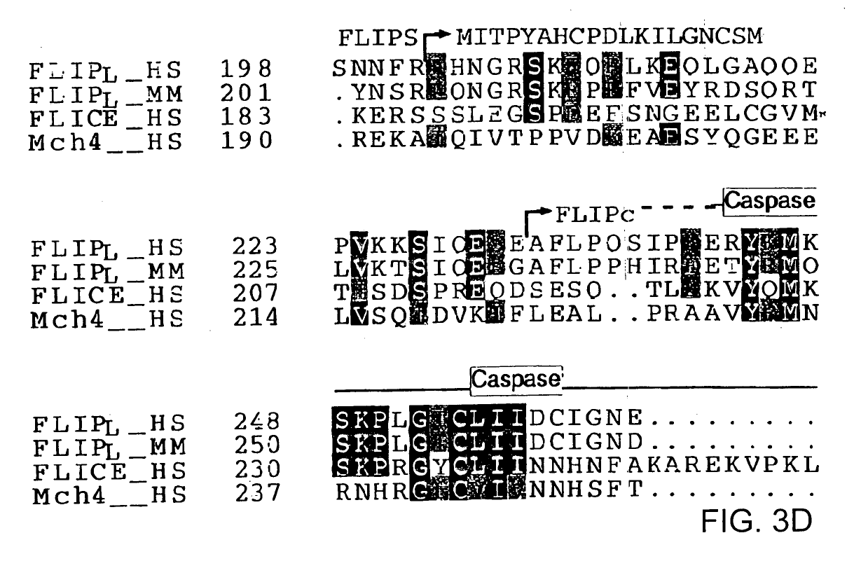

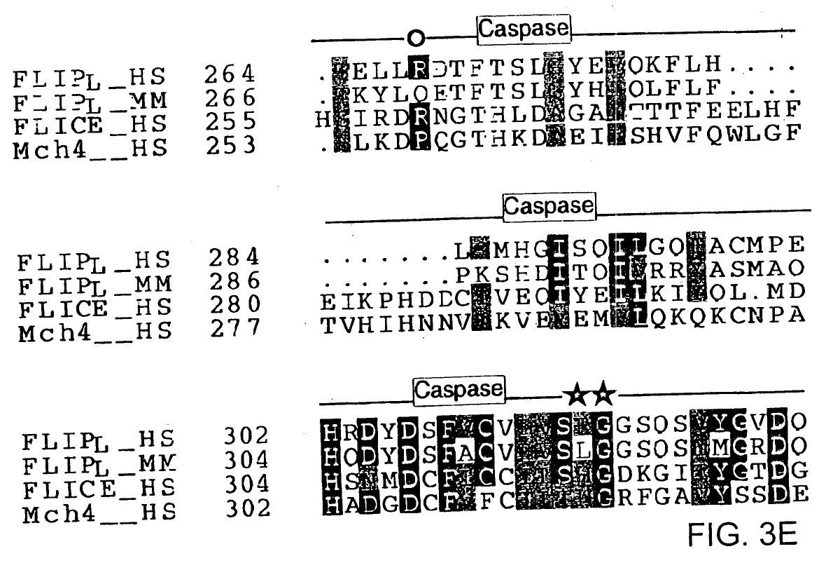

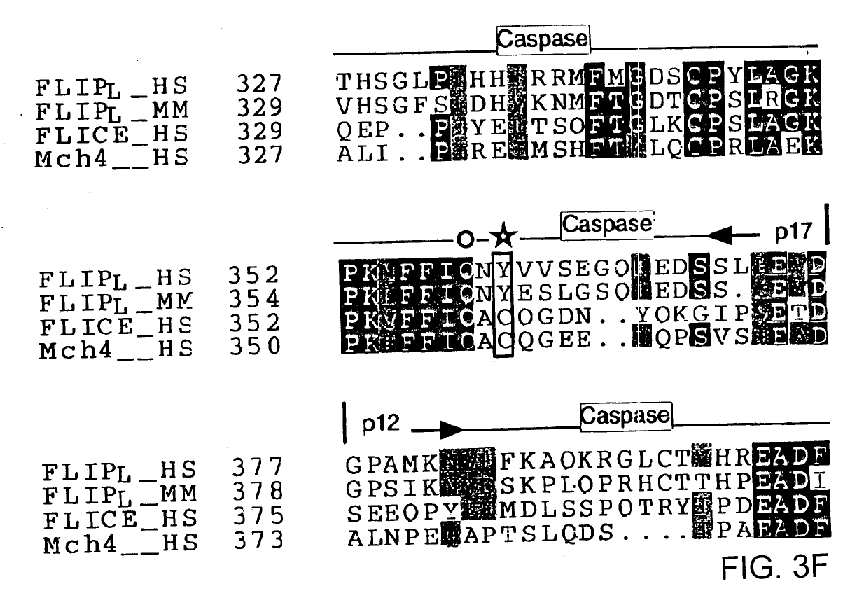

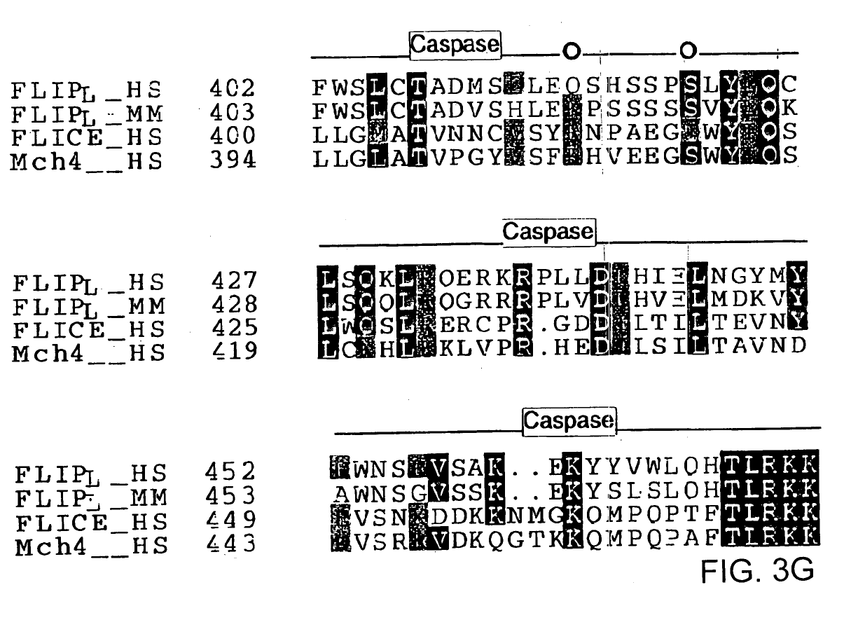

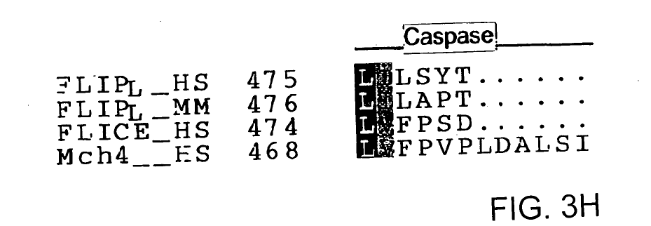

FIGS. 3a-h show that in the present sequences the cysteine residue has been substituted for a tyrosine residue. In addition, for instance as compared to the functional protease domains of FLICE, characteristic mutations occur at different sites of the caspase-homologous domain of long FLIP proteins. One might mention mutations in position −1, as related to the critical cysteine residue, and in positions +1 and +2, likewise related to the critical cysteine residue. The critical cysteine residue indicated by a star in FIGS. 3a-h, has also mutated in the mouse FLIP_{L}. In this case also, the caspase-homologous domain can therefore not fulfill any proteolytic function dependent on cysteine residue. According to this invention, non-functionality of a caspase-homologous domain is merely defined as a loss of proteolytic activity which depends on the critical cysteine residue. Even so the caspase-homologous domain may have another functionality. Such caspase-homologous domains, according to this invention, are also non-functional.

The two death effector domains of the human or murine FLIP proteins likewise interact with adapter proteins, for instance, FADD. The long protein version (55 kD) and also the short form (34 kD) associate vigorously with the FADD protein in case of a co-expression. However, the caspase-homologous sequence section of the long FLIP proteins does not show any FADD-binding affinity. Binding of a FLIP protein identified in a mammalian cell with a DNA sequence according to the invention to another protein, independent of any caspase-homologous domain, takes place via at least one death effector domain. Preferably this binding is promoted by two death effector domains. The appropriate experiments also prove that the proteins of the DNA sequences according to the invention made up of eukaryotic cells, in particular of mammalian cells including human cells, may also form a stable tri-complex with Fas/FADD. The gene products of these cellular DNA sequences according to the invention, as a result of activation of the apoptotic signal cascade, attach to the membranous death receptor domains. As soon as FADD has linked with FLIP and FAS, large aggregates insoluble even in SDS can be observed.

Without being confined to any one scientific theory, it can be determined that the caspases during their signal transmission as dimers are first activated and then processed through autocatalysis, finally forming the stable complex (p10/p20)₂. On the other hand, the long FLIP proteins made up of eukaryotic cells encoded by the DNA sequences according to the invention, in particular mammalian or human cells, do not show any dimer or oligomer formation. The FLIP proteins rather interact with the FLICE proteins in such a way that hetero-dimers or hetero-oligomers form between the active FLICE and the FLIP_{L} protein without cysteine proteolysis activity. Thus, the use of FLIP proteins with non-functional, caspase-homologous domains and with at least one death effector domain for binding to proteases, in particular to cysteine proteases functioning as elements of the apoptotic signal cascade by means of their death effector domain, is another aspect of the present invention. This binding may then block apoptotic signal transmission.

Signal cascade inhibition is based on following detailed mechanism: The FLICE protein is activated by the signal for apoptosis initiated at the death receptor, possibly by homo-dimer or homo-oligomer formation. As it is activated, either an autocatalytic fission at the p10/p20 intersection with the sequential segment of the caspase domain may occur, or the FLIP protein may bind first. In every case after the FLIP protein binds with the non-functional caspase-homologous domain, proteolysis of the FLIP protein through proteolytic activity of a FLICE protein within the FLICE/FLIP heterodimer or hetero-oligomer follows. After certain conditions which initiate apoptosis, for instance superexpression of the CD95 receptor, further proteolytic fission (processing) of the FLICE protein may occur. The FLICE protein takes on a form corresponding to a large extent to the short cellular FLIP protein (FLIP_{S}) Proteolytic fission therefore takes place at about the connection between the C-terminal death effector domain and the caspase domain of the FLICE protein.

As with the viral FLIP protein, the examples of the embodiment demonstrate that the cellular forms of the FLIP protein (human or murine, long or short FLIP protein) coded by DNA sequences according to the invention have an inhibiting effect on numerous apoptosis signals triggered by or via death receptors.

An important aspect of all DNA sequences according to the invention therefore is their utilization for immortalizing cells. This immortalization may occur in vitro or in vivo; it may involve all cell types imaginable that are capable of undergoing an apoptotic reaction. In vivo processes include all gene-therapeutic methods known to one skilled in the art.

In an embodiment of the present invention a DNA sequence according to the invention is effectively linked with a promoter. In this embodiment one promoter, preferably arranged upstream, thus binds with a DNA sequence according to the invention. This promoter may, depending on the experimental goal, be either a prokaryotic or eukaryotic promoter.

Promoters used in prokaryotic host cells might be the β-lactamase promoter or the lactose promoter, or the tryptophan promoter system or hybrid promoters, as for instance the tac promoter. The appropriate promoters are chosen by one skilled in the art, depending on the bacterial host cell. Their nucleotide sequences have been published. Through information in the literature one skilled in the art is in a position of being able to bind the promoters to the DNA sequences according to the invention (Siebenlist et al., Cell, 20: 269, 1980). Promoters in bacterial systems generally also include a Shine-Delgarno (SD) sequence.

Suitable promoter sequences in host cells can for example include the 3-phosphoglycerate-kinase promoter or promoters of other glycolytic enzymes (examples include: enolase, glycerialdehyde-3-phosphate-D-hydrokinase, hexokinase, pyruvate decarboxylase, phosphofructokinase and others).

The transcription of a DNA sequence according to the invention in cells of higher eukaryotes, in particular mammalian cells, is regulated by promoters that may be derived from differing natural systems. Thus promoters from viral genomes may be used. Examples would be polyoma viruses, SV40, adenoviruses, retroviruses, hepatitis B viruses, cytomegaloviruses and the like. With mammalian cells a possibility would be the β-actin promoter. In the current invention the Srα promoter is particularly preferred.

In particular the early promoter of the human cytomegalovirus, which contains a HindIII-E restriction site (Greenway et al., Gene, 18: 355-360 (1982) lends itself to use as a promoter of cytomegaloviruses. Of course, promoters of mammalian or human host cells can also be used.

In a particularly preferred embodiment of the invention, additional regulating elements for transcription and/or translation have been added to the DNA sequence according to the invention. Thus, the transcription of a DNA sequence according to the invention is considerably increased when an enhancer element is present in the expression vector. Enhancers are cis-acting elements of DNA which usually include 10 to 300 base pairs and act upon the promoter to raise the transcription rate. They may be arranged in the 3′ or 5′ position of the DNA sequence according to the invention, but also in the coding sequence itself or within an intron which is first cut out by splice procedures. Typically an enhancer is selected from a virus of eukaryotic cells. Examples are the SV40 enhancer or the enhancer of the early promoter of the cytomegalovirus. Enhancers of adenoviruses can also be used. Of course numerous enhancers can also be derived from mammalian genes (for instance globin, elastase, or albumin). The enhancers typically are integrated with the 3′ or 5′ position in the expression vector of a DNA sequence according to the invention. Preferably the enhancer is positioned at 5′ in relation to the promoter. Further regulating elements may serve to regulate transcription termination, so that the expression of mRNA is involved.

Another aspect of the present invention consists of expression vectors including a DNA sequence according to the invention, typically with a promoter and, if appropriate, with another of the above regulating elements of transcripton and/or translation. They serve to express and multiply the nucleotide sequence according to the invention in specific host cells. In general, expression vectors are used that can autonomously replicate independently of the host chromosome. They have their own “origin of replication.” Such sequences are present in bacteria, yeasts or viruses. On the other hand these origins are not required in the expression vectors of mammals.

If necessary the expression vectors with the DNA sequence according to the invention are developed as so-called shuttle vectors, that is, they are able to replicate in a host system and can then be transfected into another host system for purposes of expression. For instance a vector can at first be cloned in E. coli and then be inoculated into a yeast or mammalian cell for expression. In such a case it is no longer absolutely necessary for the replication to occur independently of host cell chromosome replication.

The expression vector pCR-3 or all derivatives of this vector with an EcoRI-intersection are particularly preferred. A DNA sequence according to the invention preferably is transfected into another vector, which allows stable expression in cells such as Jurkat and Raji. Typically such expression and cloning vectors include a selection gene exercising a marker function. This is a gene coding for a protein allowing host cells to survive or grow after being transformed by the vector. Typical selection genes code for proteins which permit a resistance toward antibiotics or other toxins. This, for instance, includes puromycin or ampicillin or neomycin. Within the scope of this invention, resistances to puromycin are particularly preferred with regard to selecting the transformed host cells.

An additional aspect of the present invention is a process for isolating gene products with at least one death effector domain, whereby the host cells are transformed by means of an expression vector in accordance with the invention and then cultivated under appropriate circumstances favoring the expression, so that the gene product finally may be purified out of the culture. This allows the protein of the DNA sequence according to the invention to be isolated out of a culture medium or out of cell extracts. One skilled in the art will immediately recognize that the isolation method and the purification process of the recombinant protein encoded by the DNA according to the invention in each case depends on the type of host cell, and also on whether the protein is secreted into the medium. Thus, expression systems leading to secretion of the recombinant protein may be used. In that case, the culture medium must be concentrated by means of a commercially available protein concentration filter, such as Amicon or Millipore Pelicon. After the concentration step a purification step may follow, for instance a gel filtration step. Alternatively, an ion exchanger with a DEAE matrix may also be used.

All materials known from protein purification, such as acrylamide, agaraose, dextran or similar, may be used as matrices. A cation exchanger may also be used, which typically would contain carboxymethyl groups. Further purification of a protein coded by means of a DNA according to the invention could then proceed by means of HPLC steps. Particularly the “reversed phase” method can be used. By means of these steps an essentially homogenous recombinant protein of the DNA sequence according to the invention is obtained.

Besides bacterial cell cultures for isolating the gene product, transformed yeast cells may also be used. In this case the translated protein may be secreted to simplify protein purification. Secreted recombinant protein from a yeast host cell may be obtained by methods published by Urdal et al. (J. Chromato. 296:171, 1994).

Another aspect of the present invention is a process for the expression of gene products with at least one death effector domain, whereby host cells are transformed by means of an expression vector including a DNA sequence according to the invention. The purpose of this process for expressing gene products based on a DNA sequence according to the invention is not to concentrate and purify the gene product, but rather to influence cell metabolism by means of introducing the DNA sequences according to the invention through the expression of the pertinent gene product. In this connection the use of host cells transformed by means of expression vectors for purposes of immortality must in particular be considered. Through use of a so-called constitutive promoter these cells may express constant protein concentrations based on sequences according to the invention. By this means apoptosis, either initiated by or through the mediation of, the death receptors, will permanently be prevented. The appropriate cell lines thereby become resistant to a large number of apoptotic stimuli. These cells may also be inoculated into mammalian or human organisms as the need arises. In this manner gene-therapeutic use of the DNA sequences according to the invention will be come possible by means of the cells being manipulated in the lab with expression vectors according to the invention, and their subsequent inoculation into the organism. This calls for the transfection of expression vectors with sequences according to the invention into cells which due to disease, are being eliminated as a result of apoptotic conditions in the organism.

Such a process makes possible the steady restoration of these cells endangered by apoptosis.

However, the inventive idea is also accompanied by a gene-therapeutic procedure that may be carried out in vivo. In this procedure vectors are used (for example liposomes or adenoviruses or retroviruses or the like), which insert the DNA sequences according to the invention in a specific manner into each of the organism's cells which, due to pathological conditions, exhibit an increased tendency toward cell death.

The DNA sequences according to the invention, their alleles, derivatives or fragments may also be used in the laboratory as samples. Thus, for example, the DNA sequence of the viral FLIP gene might be used to identify the appropriate viruses with in vitro test systems. This might particularly be applied with Northern or Southern blots. The samples would have the appropriate DNA sequence length depending on the requirements of the experiment. A DNA sequence on which a death effector domain of the type according to the invention was based, might for instance be used as a detection probe.

Another useful application of the DNA sequences according to the invention is their employment as single strand DNA sequences. It is possible on the RNA and on the DNA levels. Employing both the DNA and RNA of the coding strand (“sense”) and its complementary strand (“antisense”) is conceivable. Which one to use would depend on the choice of the target protein. The appropriate oligonucleotides typically include between 20 and 40 nucleotides of the DNA sequence in accordance with the invention. In this connection, the binding of anti-sense or sense DNA leads to the formation of duplex molecules either blocking translation (on the RNA level), or transcription (on the DNA level). Thus, the expression of FLIP proteins in the cell can be prevented by means of the DNA sequences according to the invention. Another possible application for this technology would result during the conception of anti-viral substances. By inoculation of these single-strand oligonucleotides, in particular the tumor-like qualities of the viruses expressing FLIP, particularly those of herpes viruses, could be combated. The oligonucleotides could be chemically altered by the appropriate modification to protect them against enzymatic decomposition. Inoculation of the oligonucleotide single strand into the cell can be performed via known methods, for instance through vectors for gene transfer, or by establishing an electronic fields.

Host cells transformed with an expression vector according to the invention are another subject of the present invention. Appropriate host cells for cloning or expressing the DNA sequences according to the invention are prokaryotic yeasts or higher eukaryotic cells. In the case of prokaryotes, gram-negative or gram-positive organisms are expressly included, such as E. coli or bacilli. E. coli 294, E. coli B and E. coli X1776 as well as E. coli W3110 strains are disclosed as the preferred host cells for cloning the DNA sequences according to the invention, and as well as bacilli, such as Bacillus subtilis, Salmonella typhimurium, and the like. As mentioned, if the expression vectors typically include a signal sequence for transporting the protein into the culture medium, prokaryotic cells can be used. Besides prokaryotes, eukaryotic microbes which have been transfected with the expression vector, are also under consideration. Thus, filamentous fungi or yeasts can be used as suitable host cells for DNA sequence-coding vectors according to the invention. Saccharomyces cerevisiae or ordinary baker's yeast (Stinchcomb et al., Nature, 282:39 (1997)) can also be used.

In a preferred embodiment, cells for expressing DNA sequences according to the invention are selected from multicellular organisms. This also takes place before a background of a possibly needed glycolysis of the coded proteins. This function may be carried out in an appropriate manner in higher eukaryotic cells as compared to prokaryotic cells. In principle every higher eukaryotic cell culture can be used as a host cell, even though cells of mammals, such as monkeys, rats, hamsters and humans, are particularly preferred. In addition, one skilled in the art is familiar with a large number of established cell lines. The following cell lines are examples of those that can be use din carrying out the invention: 293T (embryo kidney cell line) (Graham et al., Gen. Viro., 36:59 (1997)), BHK (baby hamster kidney cells), CHO (cells from hamster ovaries), (Urlaub and Chasin, P.N.A.S. (USA) 77:4216, (1980)), HELA (human cervix carcinoma cells) and other cell lines.

In accordance with the present invention, preferably cells of the mammalian immune system, above all the human immune system, have been transfected with expression vectors with DNA sequences in accordance with the invention. Here T-lymphocytes and D-lymphocytes are particularly preferred as host cells.

The case of an HIV infection demonstrates another application of the DNA sequences according to the invention which could immortalize the host cell. In an HIV infection, cells perish as a result of apoptotic mechanisms. Even non-infected CD4′ cells are subjected to cell death. To counteract an HIV infection, immortalization of the non-infected immune cells would therefore be desirable. This would allow the basic functionality of the immune system to be preserved.

Another aspect of the present invention are the gene products of the DNA sequences according to the invention. Gene products in accordance with this invention include both primary transcripts, that is, RNA, preferably mRNA, and also proteins. These proteins have at least one death effector domain and inhibit cell apoptosis. These proteins include all proteins coded according to the invention, including proteins expressed by DNA derivatives, DNA fragments, or DNA alleles. In addition, the proteins can be chemically modified. Thus, a protective group may exist at the N-terminal. Glycosyl groups may be attached to hydroxyl or amino groups, lipids may be covalently bonded to a protein according to the invention, likewise phosphates or acetyl groups, or the like. Any chemical substance, compound or group may bind to the protein according to the invention by any type of synthesis.

Additional amino acids may be fused with the N and/or C terminals, for instance as individual amino acids, as peptides, or as protein domains, and the like. In particular, so-called signal or “Leader” sequences are present at the N terminal of the amino acid sequence according to the invention, leading the peptide into a certain cell organelle or into extra-cellular space (or into the culture medium), either co-translationally or post-translationally. At the N or at the C-terminal, amino acid sequences may also be present, which due to their role as antigens, allow the amino acid sequences according to the invention to bind to antibodies. Here Flag-peptide, whose sequence in the amino acid one-letter code reads DYKDDDDK, deserves special mention. This sequence has very antigenous properties and therefore allows fast testing and easy purification of the recombinant protein Monoclonal antibodies binding to the Flag peptide are available from the Eastman Kodak Co., Scientific Imaging Systems Division, New Haven, Conn. The DNA sequences according to the invention can also be deposited on the strands of hereditary informational molecules in the form of numerous exons separated from each other by introns. Thus, all conceivable splice variants (on the mRNA-level) are likewise included among the gene products of the subject of the invention. Even the proteins encoded by these various splice variants are included in this invention.

Besides the covalent modifications of the protein, also protein aggregates, for instance dimers of the proteins or higher level aggregate, are included in the subject of the present invention.

In one embodiment, the protein has a significance level of p<10⁻² in a comparison of the sequence with a search profile according to FIG. 1a or FIG. 1b. As described above, a search profile is established to identify possible death effector domains in a data base. Two data bases are available. The data base can either be compared with a search profile, or the search profile can be compared to the data base. According to the invention, all gene products or fragments of these gene products in an embodiment are included in the subject of the invention if a primary transcript (RNA) is encoded by one of nucleic acid molecules of the invention.

In a preferred embodiment, FLIP proteins according to the invention have an amino acid sequence according to one of FIGS. 4a, 4b, or 4c (for the cellular proteins), an amino acid sequence according to FIG. 16 or 17, or GenBank access codes U60315 (MCV 159L), U60315 (MCV 160L), U20824 (E8), or X64346 (ORF 71).

Numerous applications for a protein according to the invention are disclosed. Thus, the purified protein according to the invention may, according to current usage, be administered to patients, particularly to human patients, given an appropriate pathological indication. The proteins according to the invention may freely be prepared with other proteins or pharmacological additives or physiologically acceptable carriers, or co-therapeutic materials. In this connection binding studies with other proteins are feasible. Within the scope of such binding studies the biological activity of the proteins according to the invention may be tested. A study of proteins according to the invention binding to adapter proteins such as FADD is conceivable. Application of proteins according to the invention within the scope of protein purification processes may be addressed. A protein according to the invention may be attached to a support material, such as a column, with this application serving for binding and isolating possible cell proteins physiologically interacting with the protein according to the invention.

In FIG. 1 two search profiles for identifying death effector domains have been described. In accordance with their one-letter code, the amino acids are alphabetically arranged (i.e., Alanine: A, in the first position). A systematic series of amino acids like this follows for each of the positions in the sequence. The positions in the sequence have been marked by “/M:”. This is followed by the information as to which amino acid at this sequence position has the greatest probability (SY=‘P’ for the first position in the profile sequence according to FIG. 1a), of occurring in a homologous sequence. Then, after “M=”, a systematically alphabetized series of amino acids with a probability weighing according to the profile for each of The amino acids at the sequence position specified follows. The more negative the numerical value of an amino acid, the less is the probability (in a protein having a homologous function) of finding this amino acid in this position in the sequence. In this fashion a matrix for all amino acids in all sequence positions has been constructed (see, http://expasy.hcuge.ch/txt/profile.txt, which is incorporated herein by reference in its entirety).

FIGS. 1a-1 and 1a-2 show the search profile created from the proteins FADD, Mch4, and FLICE. It includes two pages. FIGS. 1b-i and lb-2 show the search profile created from the proteins FADD, Mch4, FLICE and the viral FLIP proteins. It was used to identify the murine and human FLIP sequences. This search profile also includes two pages.

Based on these profiles, the invention also encompasses a method of locating a sequence in a database that is homologous or identical to a known sequence in a database, such as a protein database. The method includes generating a search profile based on known sequences having known biological functions.

The search profile is input into a comparison algorithm which is used to search the database to locate a sequence within the database. Preferably, the search results meet a significance level of less than 10⁻². These search profiles are machine readable, e.g., as described at http://www.expasp.ch/txt/profile.txt.

In FIGS. 2a-d the amino acid sequences of proteins known to have death effector domains (FADD, FLICE and Mch4) are compared with the amino acid sequences of viral proteins of the viruses EHV-2 (equine herpes virus 2), HHV-8 (human herpes virus 8), HVS (herpes virus Saimiri), BHV-4 (bovine herpes virus 4), and MCV (Molluscum contagiosum virus) found by means of a profile search. The sequence comparison shows homology with the first and second death effector domain (DED1 or DED2), while only a minor homology exists in the linking section between the first and second death effector domain. In the sequence comparison at hand a black background for the amino acid sign (one-letter code of amino acids, e.g., as described in Stryer, Biochemistry (1995)), corresponds to a sequence match of at least 50%, while a gray background for the amino acid sign stands for at least 50% matching through conservative amino acid substitution. The arrows at the C-terminal of the second death effector domain of the MCV 159L and MCV 160L sequences relate to their C-terminal extensions, 66 (ORF 159L) and 202 (ORF 160L) amino acids in length. The amino acid sequences of the viral FLIP_{S} have been deposited in the data base GenBank under the access code numbers U20824 (ORF E8 in EHV-2), X64346 (ORF 71 in HVS), and U60315 (ORF 159L and ORF 160L in MCV). The amino acid sequences of the viral FLIP_{S} for HHV-8 (ORF 71) and for BHV-4 have been shown in FIG. 16 (ORF 71 for HHV-8), and in FIG. 17 (v-FLIP of BHV-4). A DNA sequence coding for the amino acid sequence of the viral FLIP_{S} has been deposited in the data base GenBank under the access number U20824 (ORF E8 in EHV-2), Z46385 (BHV-4), X64346 (ORF 71 in HVS), U90534 (ORF 71 in HHV-8) and U60315 (ORF 159L and ORF 160L in MCV).

FIGS. 3a-h show the homology of the amino acid sequences of the human (HS) and the murine (MM) form of the long (FLIPL) and the short (FLIPs) version of FLIP with the amino acid sequences of FLICE and MCH4. The N-terminal 202 amino acids of the shorter splicing variant of the human FLIP (FLIPₛ) are identical with the sequence of the longer form of human FLIP (FLIP_{L}) the sequence of the shorter protein (FLIP_{S}) ends after a C-terminal extension of 19 additional amino acids (this is the only extension that has been entered for the human FLIP_{S} in this figure), which are not included in the longer form (FLIP_{L}). As do the viral FLIP_{S} (see for example the amino acid sequence of the FLIPs coded by ORF E8 of EHV-2), the murine (MM) homologue FLIP_{L} and the human (HS) homologues FLIP_{L} and FLIP_{S} both include two death effector domains each (DED1 or DED2). The murine and human FLIP_{L} form in addition have a C-terminal domain which is homologous with the protease domain of the caspases. The structural organization of FLIP_{L} (2 N-terminal death effector domains linked with a caspase) thus corresponds to the structural organization of FLICE and Mch4, but compared to the caspases the preserved cysteine residue of the active protease domain is missing: in human and in murine FLIP_{L} the corresponding amino acid position is occupied by a tyrosine residue (in the figure that position is marked by a star).

FIG. 4a shows the DNA and the amino acid sequence of the human FLIP_{S} which was isolated from a cDNA base of activated T-cells by screening with a DNA probe with a ³²P marker (EcoRI/RsaI fragment of the 5′-terminal of the DNA insert of EST clone No. 309776).

FIGS. 4b-1 and 4b-2 show the DNA and the amino acid sequence of the human C-FLIP_{L} isolated from a cDNA data base of activated T-cells by screening with the probe described in FIG. 4a.

FIGS. 4c-1, c-2, and c-3 show the cDNA and the amino acid sequence of murine c-FLIP_{L}, isolated from a murine cardiac muscle cDNA data base by screening with the probe described in FIG. 4a.

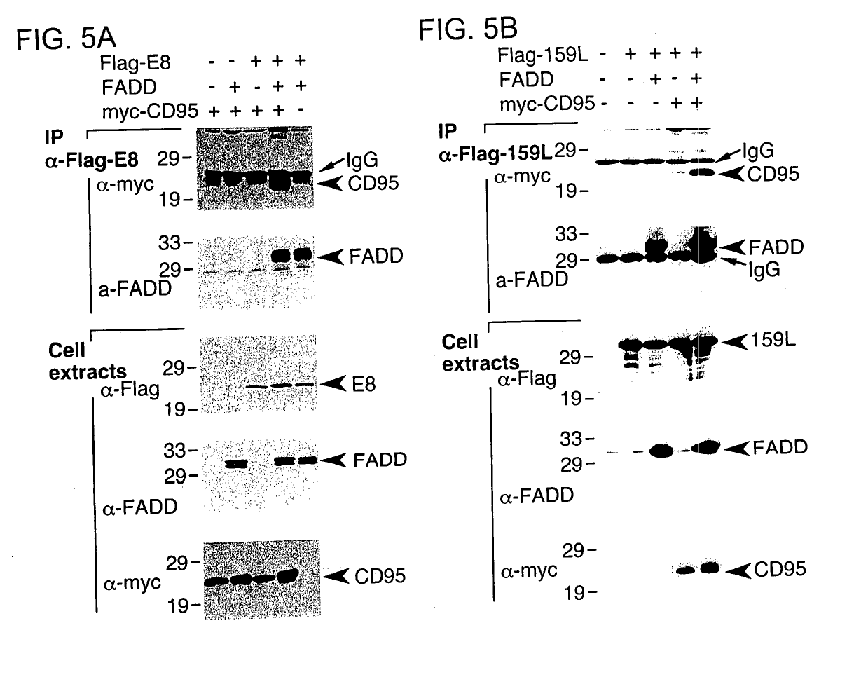

FIGS. 5a and 5b show by cotransfection experiments in eukaryotic 239T cells by the example of the viral FLIPS of EHV-2 (ORF E8, FIG. 5a) and of MCV (ORF 159L, FIG. 5b) that the viral FLIP_{S} bind to the adaptor protein FADD, and that this link does not prevent the attachment of the adaptor protein FADD to the cytoplasmic protein segment that includes the death domain of the CD95 death receptor. This binding of the viral FLIP_{S} E8 (from EHV-2) and 159L (from MCV) to FADD makes possible an attachment of the viral FLIPS, by means of the adapter FADD, to the cytoplasmic protein segment of the death receptor CD95.

The lower part of the illustrations FIGS. 5a and 5b in each case identifies the expression of the appropriate proteins (viral FLIP, FADD, and cytoplasmic protein segment of CD95 by means of the Western blot analysis described in the literature, of cell extracts for the transfected 293T-cells. In the upper part of illustration 5 the above associations (FLIP-FADD, FADD-CD95 and FLIP-FADD-CD95) are demonstrated through immune precipitations (IP). Each column corresponds to a probe, and the plus signs above the figures indicate the combination of expression vectors for the transfection of the 293T cells for each probe. In immune precipitates of Flag-marked E8 or Flag-marked 159L by means of an anti-Flag antibody, FADD can be detected in the anti-Fadd Western blot (in each case, second part of the figure from above) only if the appropriate FLIP (E8 or 159L) was co-transfected with FADD (if this is the case, it is indicated by plus signs in the first and second line of a column). An association of the viral FLIP_{S} with the myc-marked cytoplasmic protein segment of CD95 (detected by anti-myc Western blot in the uppermost part of the figure) is possible only in the presence of FADD, i.e. only if v-FLIP, FADD and the myc-marked cytoplasmic protein segment of CD95 were expressed at the same time (plus sign in all 3 lines of a column).

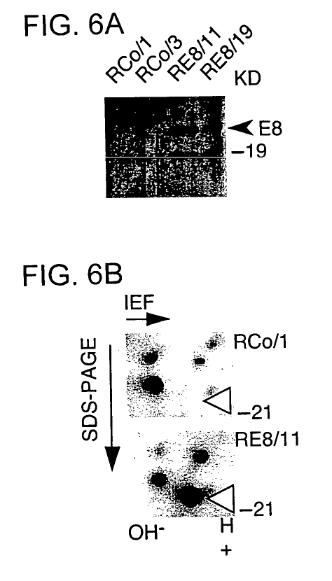

FIGS. 6a and 6b show the attachment of the viral FLIP protein E8 of EHV-2 with the agonistically stimulated CD95 receptor complex in human Raji B-cell clones, which have been stably transfected with an expression vector for the E8 protein. FIG. 6a shows the expression of the Flag-marked E8 protein in cell extracts of two Raji B-cell clones called RE8/11 and RE8/19, which were transfected with an expression vector for E8, by means of Western blot analysis. On the other hand, no E8 protein was detected in the control clones called RCo/1 and RCo/3, which were transfected with the appropriate expression vector without the insertion of E8. FIG. 6b shows the result of the analysis of immune precipitations of the CD95 death receptor stimulated agonistically with the antibody APO-1 (Kischkel et al., EMBO Journal 14, 5579-5588 (1995)) from the ³⁵S metabolically marked Raji cell clones Rco/1 (a control clone not expressing E8). A white arrowhead marks the migration position of the radioactively marked E8 protein that is included in the CD95 attachment complex of the RE8/11 clone, but not in the corresponding complex of the RCo/1 control clone.

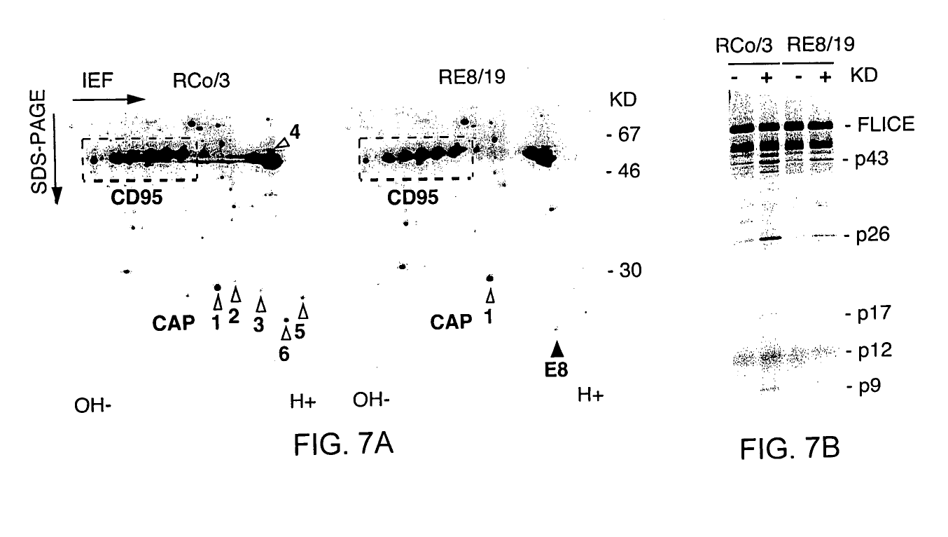

In FIG. 7a the association of ³⁵S-marked proteins with the CD95 death receptor stimulated agonistically (Kischkel et al., EMBO Journal 14, 5579-5588 (1995)) in the non-transfected Raji control cells (clone Rco/3, left part of the illustration) is compared by two-dimensional gel electrophoresis analysis of CD95 (anti-APO-1) immune precipitations to the same association for such cells that were transfected with an expression vector for the viral FLIP E8 of the EHV-2 (clone RE8/19, right part of the illustration). The figure shows that the viral protein E8 associates with the agonistically stimulated CD95 death receptor without interfering with the attachment of the adaptor molecule FADD (CAP 1) to the receptor (the FADD protein called CAP1 is included in the agonistically stimulated CD95-associated signal transduction complex of the control clone RC0/# that does not express E8, and also in the Raji clone RE8/19 which does express E8; compare right and left part of the figure). The figure further shows that in the cells expressing the viral FLIP, the attachment of FLICE (CAP4) and of FLICE-like molecules (CAP3) to the receptor is prevented, and therefore the conversion of FLICE into its two fission products CAP5 and CAP6 taking place in the receptor attachment complex is blocked (absence of the proteins with radioactive markers 4-6 in clone RE8/19 expressing E8 only in the right, but not in the left part of the figure).

FIG. 7b shows that the FLICE fission activity of the agonistically stimulated receptor of the Raji B-cell clone RE8/19 which expresses E8, induced by treatment of cells with the agonistically stimulating anti-CD95 antibody APO-1, is considerably less than the corresponding activity of the control clone Rco/3 (which does not express E8). The presence of FLICE fission activity in the CD95 attachment complex is quantifiable through the formation of the FLICE fission products p43, p26, p17, p12, and p9 from −42-radioactively marked FLICE by means of CD95 (anti-APO-1) immune precipitate of APO-1-treated (+) cells, but practically not at all of untreated (−) cells. In the FLICE fission experiment the bands for the molecular mass described above are 43, 26, 17, 12, and 9 kD for the APO-1-stimulated (+), but practically not at all for the unstimulated (−) Raji control clone RC0′3, which does not express E8. On the other hand, the fission activity of the CD95 attachment complex of the clone RE8/19 expressing E8 induced by APO-1 treatment is much reduced (the corresponding FLICE fission products are almost undetectable for this clone). FIG. 7 therefore shows that the presence of the FLIP E8 from EHV-2 in the DISC of the death receptor CD95 impedes the attachment of FLICE as well as its activation caused by proteolytic fission.

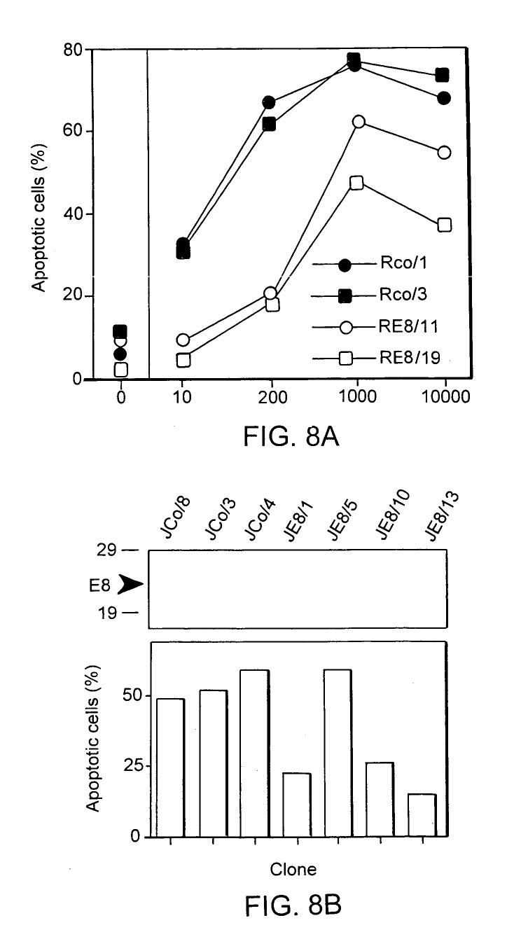

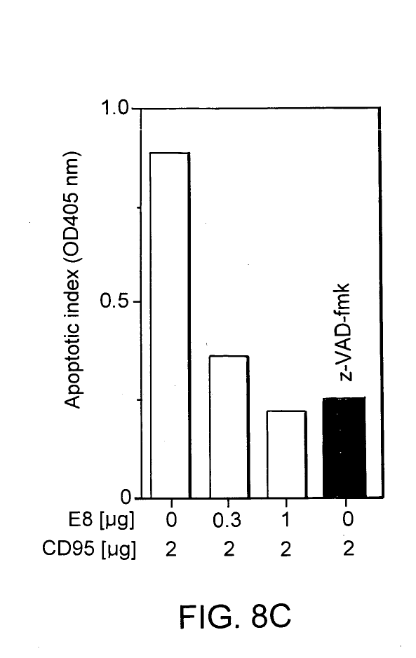

FIGS. 8a, b, and c show that through expression of a viral FLIP (ORF E8 of EHV-2 or ORF 159L of MCV) eukaryotic cells become considerably more resistant to apoptosis induction by the CD95 death receptor than do control cells. FIG. 8a shows the relationship of the percentage of the apoptotic Raji B-cells (Y-axis) to the concentration (in the culture medium for 24 hours at 34° C.) of the agonistic anti-CD95 (APO-1)-antibody (X-axis) for two Raji control clones not expressing E8 (Rco/1 and RDp/3). The two clones expressing E8 proved distinctly more resistant to the CD95-apoptosis than the control clone. A APO-1 concentration in the culture medium of 100 ng/ml was sufficient under the selected experimental conditions to induce apoptosis in over 60% of the control clones not expressing E8, while under the same conditions only about 20% of the cells of the two Raji clones RE8/11 and RE8/19 expressing E8 were apoptotic.

Inducing apoptosis in ca. 50% of the Raji Raji clones RE8/11 and RE8/19 expressing E8 required a more than 10 times higher APO-1 concentration was required than for the of the control clones RCo/1 and Rco/3 not expressing E8.

FIG. 8b shows that through expression of the viral FLIP of the E8 ORF of EHV-2, human Jurkat T-cells will also acquire resistance against induction of apoptosis via the CD95 death receptor. The experiments were conducted with E8 expressing Jurkat clones (JE8/1, JE8/10 and JE8/13 and a Jurkat clone JE8/5 not expressing E8, as well as control vector-transfected clones (yjCo/2 and JCo/4). The sensitivity of the clones toward CD95 ligand-induced apoptosis was determined by means of incubation of the clones for 3 hours at 370 with supernatant of CD95L-producing neuronal cells diluted 1/10 in a culture medium (Rensing-Eh et al., Eur. J. Immunol. 25, 2253-2258 (1995)), and subsequent analysis of the propidiumiodide marked cells in the FACScan flow-through cytometer (Nicoletti et al., J. Immunol. Methods 139, 271-279 (1991)). FIG. 8b shows in particular that the among of the viral FLIP_{S} marked with Flag expressed in the clones (identified by the Western blot described in the literature, of cell extracts with anti-Flag antibodies, upper section of FIG. 8b) correlates with a reduction of the apoptosis sensitivity toward the CD95 ligand (lower section of FIG. 8b), for the percentage of the apoptotic cells induced by the CD95 ligand (as indicated on the X-axis) is the lower, the more vigorous the E8-expression of the clone detected by the Western blot in the upper section of FIG. 8b. In particular, under the experimental conditions chosen, the percentage of the apoptotic cells with control clones not expressing E8 (JCo/2, JCo/3, JCo/4 and JE8/5) was at least three times as high as the percentage of the apoptotic cells for the Jurkat clone with the most vigorous E8 expression (clone JE8/13).

FIG. 8c shows the shielding effect of vFLIPs (it shows an experiment with the gene product of ORF E8 from EHV-2) with induced apoptosis by means of over-expression of the death receptor CD95 in 293T-cells (a human embryo kidney cell line). For this experiment 293T cells with expression vectors for CD95 were transfected together with the quantities of expression vectors stated for the above-mentioned V-FLIP in FIG. 8c and harvested 30 h after transfection. The apoptosis induction in the cells was determined through photometric quantification of apoptotic histone-DNA complexes (the apoptotic index stated on the Y-axis correlates with the optical density (OD) of the samples at 405 nm as determined with the Elisa cell-death detection system by Boehringer). Cells transfected with expression vector without insertion (mock), or transfected with the expression vector for CD95, after transfection were incubated in medium with 25 μM z-VAD-fmk until harvested, served as negative controls with apoptosis induction. The figure shows that the gene product of E8 of the EHV-2 can shield 293T cells, from apoptosis induction through CD95 over-expression, whereby the shielding effect rises with the concentration of the v-Flip expression vector (this corresponds to a lowering of the apoptotic index with the rising v-FLIP expression vector concentration in FIG. 8c). With the highest doses used (co-expression of 1 μg each of the expression vectors for E8 with the amount of the CD95 expression vector stated in FIG. 8c), the shielding effect of the viral FLIP_{S} is comparable to the protection achieved through the protease inhibitor z-VAD-fmk.

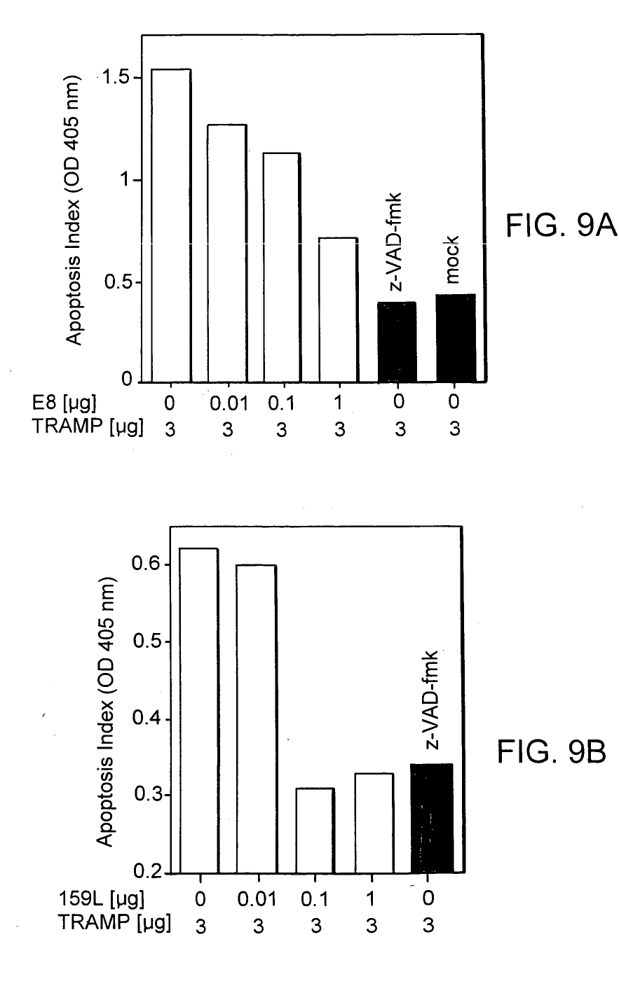

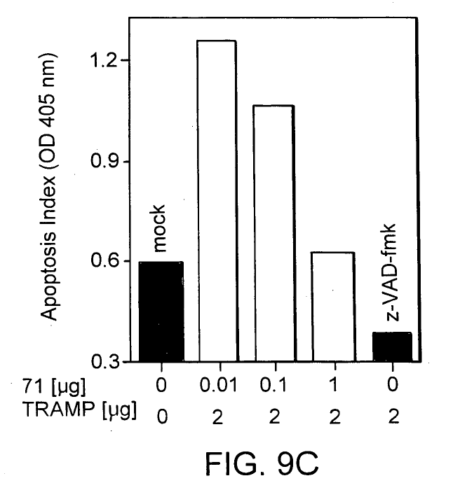

FIGS. 9a, b, and c show the shielding effect of v-FLIPs (shown are experiments with the gene products of the ORF E8 of EHV-2, ORF 159L of MCV and ORF 71 of HVS) on induced apoptosis by over-expression of the death receptor TRAMP in 293T-cells (a human embryo kidney cell line). With these experiments 293 T cells with expression vectors for TRAMP (Bodmer et al., Immunity 6, 79-88 (1997)) were transfected together with the indicated quantities of expression vectors for the above v-FLIP_{S} and harvested 30 hours after transfection. Cells were transfected with an expression vector without insertion (mock), or were transfected with the expression vector for CD95, and after transfection were incubated in medium with 25 μM z-VAD-fmk until harvested, served as negative controls for the apoptosis induction. The apoptosis induction in the cells was determined as described in FIG. 8c. For all three of the v-FLIP_{S} shown, with increasing concentration of the v-FLIP expression vector there is a reduction of the apoptosis induced by TRAMP over-expression (quantified via the optical density at 405 nm as described above). For each of the greatest quantities of the v-FLIP expression vectors used in the experiments (the expression vector quantity has been stated below the figures in μg) an apoptosis shield of the TRAMP over expressing cells of at least 70% was achieved (as compared to 100% protection through the protease inhibitor z-VAD-fmk).

FIG. 10 shows that the human Jurkat T-cells through expression of viral FLIP of the E8 ORF of EHV-2, will acquire resistance against induction of apoptosis via the receptor for death ligand TRAIL. Here the quantity of the viral FLIP expressed in the clones (as determined by Western blot test on cell extracts, upper section of FIG. 8b) correlates with the amount of cell viability in the presence of the apoptosis-inducing death receptor ligand TRAIL (FIG. 10). The sensitivity of the above-mentioned clones toward TRAIL-induced apoptosis was determined by means of incubation with the concentrations of recombinant soluble Flag-marked TRAIL and cross-linked anti-Flag antibody for 20 hours at 37° and subsequent determination of the proliferation rate of the cells (Y-axis) by means of a cell profile assay (Cell titer 96 AQ, Promega). With increasing TRAIL concentration the Jurkat clones not expressing E8 (JCo/2, JCo/3, JCo/4 and JE8/5) in this test showed a definite reduction in proliferation (sigmoid curve, reduction of optic density with decrease in proliferation). With Jurkat clone JE8/13, on the other hand, with the most vigorous E8 expression, the observed cell proliferation remained nearly the same (horizontal curve). The two Jurkat clones with an intermediate E8 expression level (J8/1 and J8/10 showed only a slight proliferation decrease due to the effect of being treated with TRAIL.

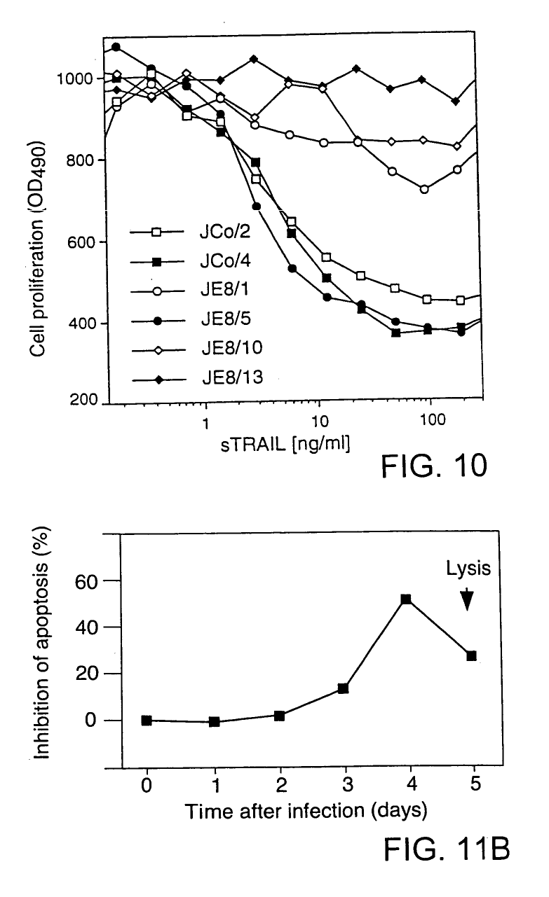

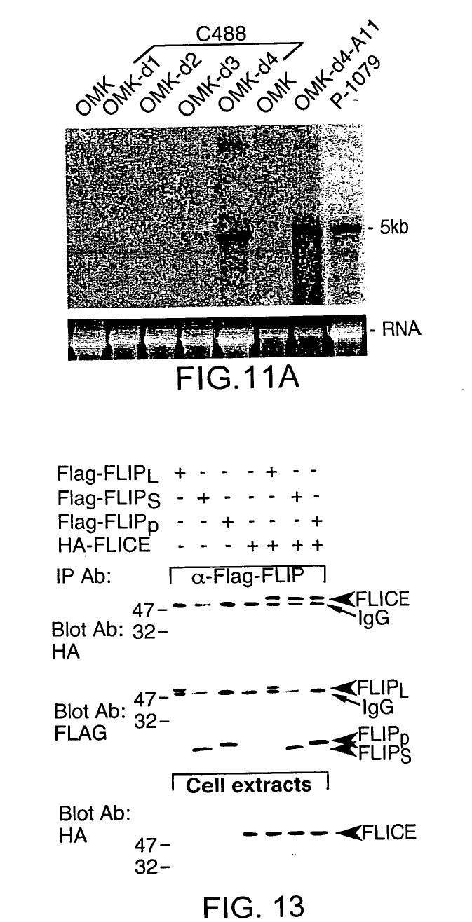

FIG. 1ia and b demonstrate the correlation of the expression of a viral FLIP (OrF 71 of HVS) in the course of the viral infection of the host cell OMK (owl monkey kidney) with the shield of the host cell against CD95 ligand induced apoptosis. FIG. 11a shows a Northern blot analysis for detecting transcripts of ORF 71 of HVS in probes of non-virus-infected control cells (OMK), in probes of OMK cells, infected with the HVS strain C488 for 4 days (C488, OMK d1-d4), or were infected with the HVS strain All for 4 days (OMK d4-Al), or of probes of a semi-permissive T-cell line producing small amounts of virus particles (P-1079). In virus-infected OMK cells a specific transcript of ca 5 kb can be detected on the fourth day of the infection. This correlates with the inhibition of the CD95 ligand induced apoptosis of virus infected cells shown in FIG. 11b as compared to non-infected cells at this time. The seemingly slight apoptosis protection on day 5 of the virus infection is due to the onset, at this time, of a massive cell lysis due to infection (ca. 50% lysis on day 5 compared to less than 10% lysis on day 4). Inhibition of the CD95 ligand-induced apoptosis of virus-infected OMK cells as compared to non-infected control cells was determined through incubation of the cells with recombinant CD95 ligand and cross-linked anti-Flag antibody for 20 h and subsequent quantification of apoptotic histone-DNA complexes (Cell-death detection ELISA, Boehringer).

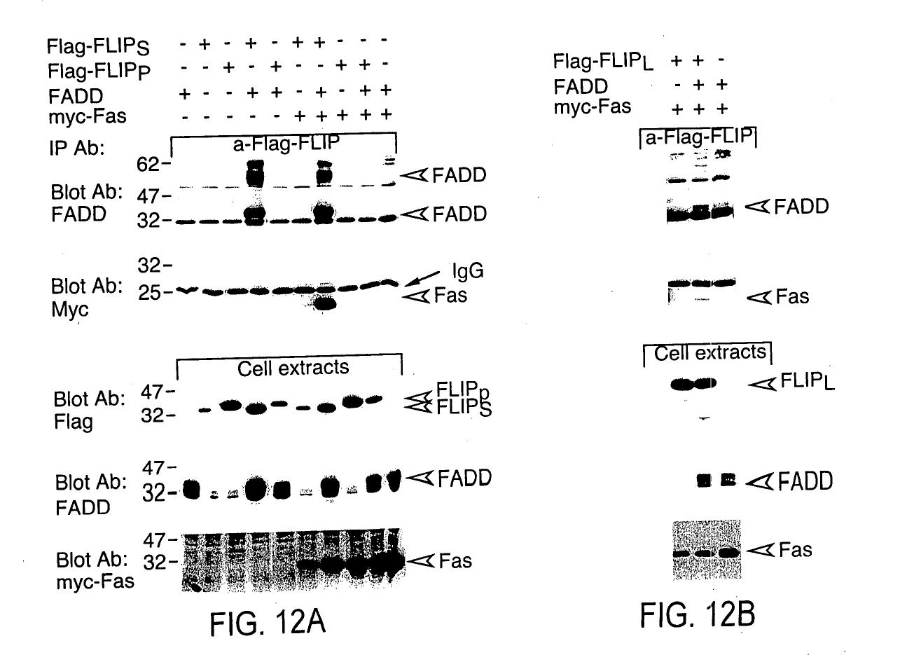

FIGS. 12a and b show through co-transfection experiments in eukaryotic 293 T cells that the short and long form of human FLIP, (FLIP_{S} and FLIP_{L}), but not the protein segment with c-p-ase homology (FLIP_{L}) will bind to the adapter protein FADD. The figure further shows that the linkage of FLIP_{S} and FLIP_{L} to the adaptor protein FADD, does not prevent the attachment of FADD to the cytoplasmic protein segment of the CD95 death receptor which includes the death domain. By means of the linkage of human FLIPS and FLIP_{L} to the FADD adapter protein an attachment of these FLIP forms to the cytoplasmic protein segment of the CD95 death receptor is made possible via the adapter FADD.

In the lower part of FIGS. 12a and 12b the expression of the appropriate proteins (FLIPₛ, FLIPₚ, FLIP_{L} FLIP_{L} FADD and the cytoplasmic protein segment of CD95) in each case is verified through Western blot analysis of cell extracts of the transfected 293T cells, while in the upper part of the FIGS. 12a and 12b the protein associations described above (FLIPs, or FLIP_{L} with FADD, FADD with CD95 and FLIP_{S} or FLIP_{L} via FADD with CD95) are demonstrated through immune precipitations (IP). Every column here corresponds to a probe, and plus signs above figure sections 12a and 12b characterize the combination of expression vectors for the transfection of the 293T cells. Within immune precipitations of Flag-marked human FLIP by means of an anti-Flag antibody the forms of the human FLIP_{S} (either FLIP_{L} or FLIP_{S}), for the two N-terminal death effector domains, and FADD can be detected in the anti-FADD Western blot (uppermost part of the figure) only if a FADD expression vector was used during transfection (plus sign in the appropriate lines of the column). The caspase-homologous protein segment (FLIP_{P}) of the human FLIP_{S} which does not include the two death effector domains cannot associate with FADD (result of the anti-FADD Western blot of the probes in columns 5 and 9 in FIG. 12a). An association of the forms FLIP_{S} or FLIP_{L}, but not the protein segment of FLIP_{P}, with the myc-marked cytoplasmic protein segment of CD95 (detected by anti-myc Western blot, see second part of the figure from the top) is possible only in the presence of FADD, i.e. only if FLIP_{S} or FLIP_{L}, FADD and the myc-marked cytoplasmic protein segment of CD95 were expressed together (column 7 of FIG. 12a and column 2 of FIG. 12b).

FIG. 13 shows by cotransfection experiments in eukaryotic 239T cells that the human FLIP_{S} and FLIP_{L} bind to the cysteine protease FLICE. The figure further shows that both the N-terminal protein segment including the death effector domains of FLIP_{L} and also the caspase homology endowed C-terminal protein segment of FLIP_{L} contribute to binding FLIP_{L} to FLICE. Expression of the HA-marked FLICE protein in cell extracts of the transfected probes was determined in the lower-most part of the figure by means of a Western blot test with anti-HA-antibodies. The central portion of the illustration by means of an anti-Flag Western blot shows that comparable quantities of three different Flag-marked FLIP proteins or protein segments were precipitated out of the transfected cells by means of anti-Flag immune precipitation. The uppermost figure portion, by means of an anti-HA Western blot analysis of the anti-Flag precipitates, that the HA-marked FLICE binds to FLIP_{L} and FLIP_{S} and also to the caspase-homologous C-terminal protein segment of FLIP_{L} (FLIP_{P}).

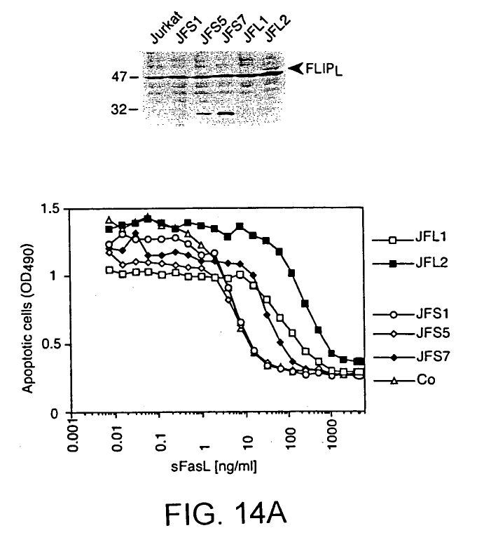

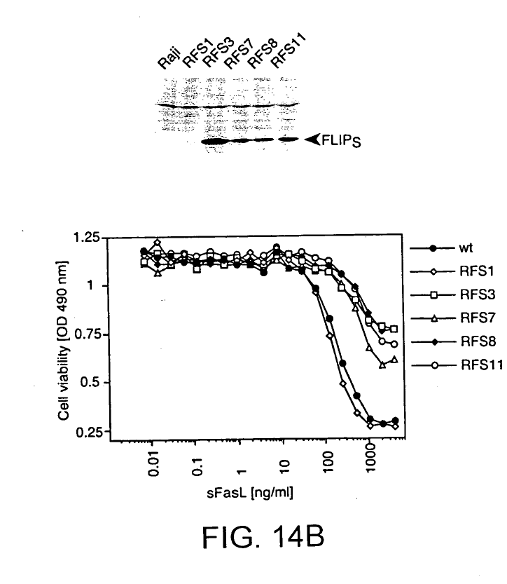

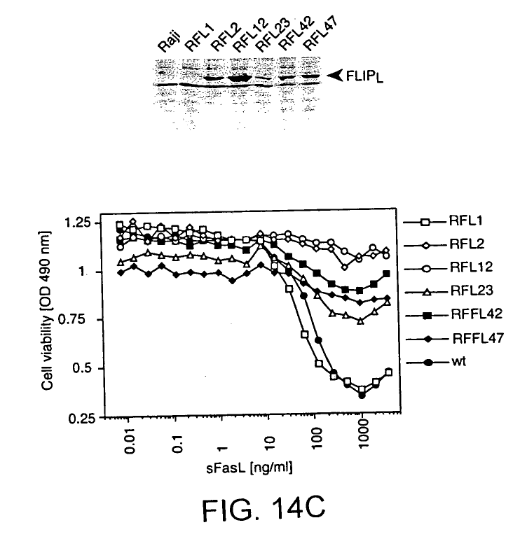

FIGS. 14a, b, and c shows that through expression of a human FLIP_{S} or FLIP_{L} eukaryotic cells become considerably more resistant to apoptosis induction via the CD95 death receptor than do control cells. It also demonstrates that the longer form of the human FLIP (FLIP_{L}) provides more effective protection against CD95-induced Apoptosis than the shorter form FLIP_{S}. FIG. 14a shows that human Jurkat T-cells through expression of the human FLIP_{S} or FLIP_{L} will also acquire resistance against induction of apoptosis through the Fas(CD95) ligand (FaslL). Here the quantity of the human VSV-marked FLIP_{S} or FLIP_{L} expressed in the clones (as determined by Western blot test of cell extracts, upper section of FIG. 14a) correlates with the amount of cell viability in the presence of the apoptosis-induced FasL (lower part of FIG. 14a). The sensitivity of the Jurkat clones towards FasL-induced apoptosis was determined by incubation with the concentrations stated for the X-axis of recombinant soluble flag-marked FasL (sFasL) and cross-linked anti-Flag antibodies for 20 hours at 37° C. and subsequent determination of the proliferative ability of the cells (Y-axis), by means of a cell proliferation test (cell titer 96 AQ, Promega). The parental Jurkat T-cells not transfected (Jurkat, Co) the non-FLIPₛ expressing Jurkat clone JFS1 and the poorly FLIPₛ-expressing Jurkat clone JFS5 in this test show an overlapping sigmoid curve (decrease of proliferation with increase in sFasL concentration). On the other hand, a shift to the right JFS7 was observed with the proliferation curve for the more vigorous FLIPs-expressing clone. To achieve a decrease in proliferation comparable to that of the parental control clone with clone JFS7, an approximately 5 times higher FasL concentration was needed as compared to the parental clone. An even more vigorous resistance against FasL was observed for the two FLIP_{L}-transfected Jurkat clones JFL1 and JFL2. Although the protein expression of the VSV-marked FLIP_{S} was merely weak (JFL2) or did not show up in the Western blot at all (JFL1), 48 (upper part of FIG. 14a) the two clones were approximately 10 times (JFL1) or more than 25 times (JFL2) more resistant against treatment with sFASL than the parental Jurkat clone.

FIG. 14b shows that also human Raji B-Cells through expression of a human FLIP_{S} become considerably store resistant to apoptosis induction via sFas1. For the experiments the Raji clones RFS3, RFS7, RFS8, and RFS11, expressing the human FLIPS, and a Raji clone not expressing the human FLIP_{S}(RFS1) and the non-transfected parental Raji clone (Raji wild type wt) were used and the sensitivity of the clones to sFasL-induced apoptosis determined as in FIG. 14a. FIG. 14b shows that Raji clone expressing VSV-marked FLIP_{S} (see anti-VSV western blot in the upper part of FIG. 14b) show a partial resistance against sFasL. Under the selected experimental conditions these clones were at least 5 times more resistant against the obstruction to proliferation than the non FLIPS-expressing control clones Raji (wt) and RFS1.

FIG. 14c finally shows by means of the experimental approach described with FIG. 14a that human Raji B cells through expression of human FLIP_{L} may acquire a practically total resistance to treatment with sFasL. In particular the Raji clones with a vigorous (RFL12) and medium (RFL2, RFL42, and RFL47) expression level of the VSV-marked FLIP_{L} were almost completely resistant against treatment with sFasL at the concentrations shown for sFasL in the X-axis (this showed in a nearly horizontal curve, indicating unchanged cell proliferation behavior of the clone in question, even during high (above 1 μg/ml) sFasL concentrations).

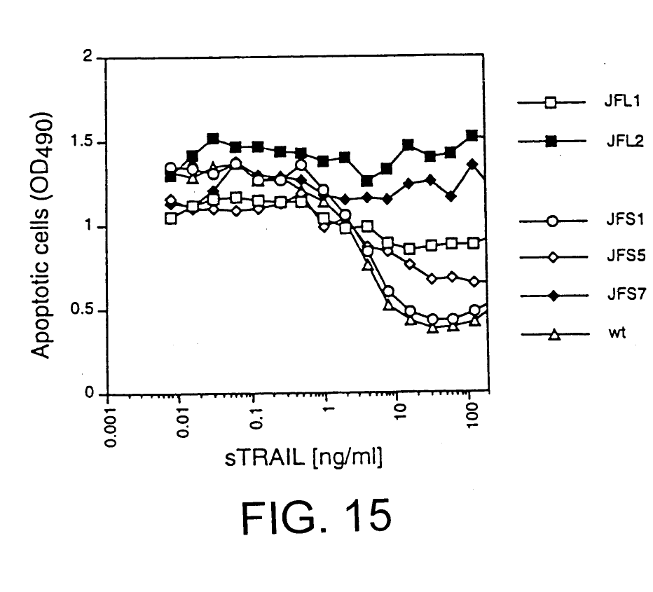

FIG. 15 shows that human Jurkat T cells acquire resistance to apoptosis induction by the receptor for the death ligand TRAIL through expression of the human FLIP_{S} or FLIP_{L}. Here the quantity of the c-FLIP_{S} expressed in the clones (as determined by Western blot test on cell extracts, upper section of FIG. 14a) correlates with the amount of cell viability in the presence of the apoptosis-inducing death receptor ligand TRAIL (FIG. 15). The sensitivity of the Jurkat clone toward TRAIL-induced apoptosis was determined by through incubation with the concentrations given in the X-axis of recombinant soluble Flag-marked TRAIL and cross-linked anti-Flag antibody for 20 hours at 37° C. and subsequent determination of the proliferation rate of the cells (Y-axis) by means of a cell proliferation assay (Cell titer 96 AQ, Promega). With increasing TRAIL concentration the parental non-transfected Jurkat clone (wt) and the non-transfected, non-FLIPS-expressing clone JFS1 in this test showed a definite reduction in proliferation (sigmoid curve, reduction of optic density with decrease in proliferation). Clone JFS5, which in the Western Blot (FIG. 14a above) showed an intermediate level of VSV-marked FLIPs, showed partial resistance to TRAIL, while clone JFS7, which expresses the VSV-marked FLIP_{S} more vigorously, was practically completely resistant against TRAIL. The horizontal curve shows that no reduction of proliferation through death receptor ligand TRAIL occurred). The experimental curves also show that the two Jurkat clones JFL1 and JFL2 transfected with an expression vector for human FLIP_{L} in spite of a very weak (clone JFL1) or weak (clone JFL2) expression of the VSV-marked FLIP_{L} (see Western blot test FIG. 14a, upper part) were almost (JFL1) or nearly (JFL2) resistant against treatment with the death receptor ligand TRAIL.

FIG. 16 shows the amino acid sequence of the viral FLIP_{S} of HHV-8 (ORF 71) in the one-letter code of the amino acids. A DNA sequence coding for this amino acid sequence is found in the data base GenBank with the access number U90534 (OEF 71 in HHV-8).

FIG. 17 shows the amino acid sequence of the viral FLIP of BHV-4 in the one-letter code of the amino acids. A DNA sequence coding for this amido acid sequence can be found in the data base GenBank under access code Z46385.

### EXAMPLES

The present invention is further explained by means of the following non-limiting examples. First we describe the experimental framework for all of the following examples.

The cell lines used were a human embryo kidney cell line (293T cells), a human leukemia T-cell line (Jurkat cells), or a human Burkitt lymphoma B cell line (Raji cells) and cultivated as described with Bodmer et al. (Immunity 6, 79-88 (1997)).

The monoclonal antibodies used for the immune precipitation and the “Western blotting” were anti-Flag antibodies and anti-Flag agarose (by Kodak International Bio-technologies), and anti-FADD antibodies (from Transduction Laboratories), and antibodies against the myc-Epitope (from the Sigma company, 9E10), against the VSV-Epitope (from the Boehringer company) and against the HA-epitope.

A soluble component of the human TRAI protein, indeed with the amino acids 95-281, was produced from an EST (Expressed Sequence Tag) clone through a PCR-process. The clone is called 117926 and has been entered in the GenBank under access code T90422. The following sequences were used as oligonucleotides: the oligonucleotide JT403 5′-TCAGCTGCAGACCTCTGAGGAAAC-3′ and the oligonucleotide JT469 5′-ACTAGTTAGCCAACTAAAAA-3′. The section was cloned in the vector pQE-16 (by the Qiagen company) in a PstI/speI intersection—depending on restriction enzyme abbreviations. The cloned sequence also includes a Flag sequence and a connecting element following this, with the amino acid sequence GPGQVQLQ. This is followed by PstI and SpeI intersections between the original BamHI/XbaI sites of the vector. Protein expression in the bacteria was induced with 0.5 mM IPTG. After 6 hours of incubation at 300, the cells were harvested and lysed by means of sonication. The lysates were first extracted with 0.5% pre-condensed triton x-114 in order to eliminate the bacterial lipopolysaccharides, and finally the Flag/TRAIL protein was cleared with Me anti-Flag agarose by means of a chromatography column; after that elution with 50 mM citric acid and finally neutralization with a 1 M Tris base and dialysis against PBS (phosphate buffered saline).

For isolating the cDNA clones of the human and murine FLIP, a DNA fragment of the human FLIP DNA sequence included in EST clone 309776, and obtained by PCR amplification was used as a ³²P marked probe. This fragment corresponds to the DNA sequence section numbered 394 to 903 in FIG. 4a. The screening of the λ-ZAP cDNA database of activated human lymphocytes (Stratagene, available upon request through Hermann Eibel, U. of Freiburg, Germany) for isolating the human forms of FLIP and the screening of a bank of murine cardiac cells (Stratagene) for isolating murine FLIP_{L} was carried out according to the manufacturer's directions.

The complete Open Reading Frame E8 of virus EHV-2 (ORF E8) was amplified from viral DNA by PCR methods, whereby a 5′primer with an EcoRI sequence extension and a 3′primer with a sequence extension for the restriction enzymes BamHI and EcoRI were used. The insertion (into the EcoRI intersection of the vector) occurred in the same reading frame as with the N-terminal Flag Epitope. The vector was derived from pCR-3-vector (from the Invitrogen company). The open reading frames procedure was used analogously with ORF 159 of virus MCV (from the Institut für Medizinische Virologie der Universitãt Heidelberg, Germany) and the Open Reading Frame ORF 71 of the virus HVS. The FLIP-coding ORFs of these viruses were also amplified through PCR methods and then inserted in the correct reading frame with the N-terminal Flag-Epitope into the into the EcoRI intersection of the vector derived from pCR-3. Further, the complete ORF of the human FLIP_{S} and the human FLIP_{L} and also a HindIII/XhoI fragment of the 3′ terminal of the DNA sequence of the human FLIP_{L} were amplified by PCR methods and cloned in the correct reading frame in vectors derived from pCR-3, which add an N-terminal Flag or VSV Epitope to the gene products in question. In order to achieve a stable expression of FLAG E8, VSV-FLIP_{S} and VSV-FLIP_{L} in Jurkat and Raji cells, the constructs from Flag-E8, VSV-FLIP_{S} and VSV-FLIP_{L} were further cloned in the multiple cloning site (MCS) of the vector pSRαpuro (a gift from R. Sekaly, IRCM, Montreal, Canada). This vector exhibits puromycin resistance.

An expression vector for the cytoplasmic domain of the murine FAS receptor provided with the myc sequence was produced by insertion of a PCR fragment corresponding to the amino acids 166-306, in the correct reading frame with the N-terminal myc-Epitope, and was inserted in the pCR-3 type vector. The expression vector for the human TRAMP or FADD in Vector pCR-3 can be found in the publication by Bodmer et al (Immunity 6, 79-88 (1997).

Human Fas (with the nucleotide sequence −24 to +1009) was amplified from an EST clone by PCR methods (GenBank access code X63717) and then subcloned into the pCR-3 vector as a HindIII/XbaI fragment. In order to obtain stable puromycin-resistant transfectants of Jurkat and Raji cells, the cells were washed with HeBS-buf fer with a pH of 7.05 (0.8 mM NaH₂PO₄.2H₂O), 20 mM Hepes, 137 mM NaCl, 5 mM KCl, and 5.5 mM D-glucose). Then 8×10⁶ cells were resuspended in 800 μl, were mixed with 20 μg of the SRαpuro plasmids (with and without Flag-E8 insertion) and finally exposed to a voltage of 250 V and a current of 960 μF. After 48 hours of transfection the cells were distributed on flat-bottom plates at a concentration of between 2,000 to 20,000 cells each well and stable transfectants selected by adding 5 μg/ml puromycin (from Sigma Co).

To achieve a temporary transfection of 293T cells, the cells were distributed at a concentration of 1 to 2×10⁶ cells/10 cm plate or 3 to 2×10⁵ cells/5 cm plate and transfected the following day with the aid of the calcium phosphate precipitation method described in the literature. The precipitate was left on the cells for 8 hours and the cells were finally harvested 26 to 30 hours after transfection.

The 293 T cells of a 10 cm plate were lysed after transfection in 200 μl lysing buffer (with 1% NP40, 20 mM Tris-HCl, pH7.4, 150 mM NaCl, whereby in addition 1 mM EGTA, 1 mM pefabloc-sc (from the Serva Co.) and in each case, 10 μg/ml leupeptin and aprotinin (from the Sigma Co.) were added. Post-nuclear lysates were pre-purified for at leased 1 hour before precipitation on Sepharose 6B (from Pharmacia). Subsequently immuno-precipitation was undertaken with 3μ. anti-Flag Agarose, either for two hours or overnight. The precipitates were washed in lysing buffer for a total of four times, with the lysing buffer during the first two washings containing 1% Np40 and during the last two washings 0.1% NP40. The precipitates were then heated in probe buffers and analyzed with SDS-PAGE and Western blotting. The blots were then saturated with 5% milk in PBS with 0.5% Tween. They were then incubated at room temperature for one hour using monoclonal anti-Flag-antibody at a concentration of 5 μg/ml, with monoclonal anti-myc antibody (9E10) at a concentration of 5 μg/ml, or with monoclonal anti-HA antibody at 1 μg/ml, whereby a second antibody marked with Peroxidase was added (second antibody from the Jackson Laboratories). The detection of the proteins was enhanced by chemi-luminescence (Amersham International). Jurkat, Raji or 293 cell clones were tested for their expression of transfected proteins. This was done with anti-Flag, anti-VSV, anti-FADD, anti-myc, or anti-HA Western blot analysis of post-nuclear cell lysates of equivalent protein contents. The metabolic marking of Raji cells with ³⁵S, the anti-CD95 immune precipitation and their 2D-gel electrophoresis were conducted as described above (Kischkel, F. C. et al., EMBO J. 14, 5579-5588 (1995)).

The analysis of the apoptosis induced by FasL (DD95L) was undertaken as follows. Puromycin-resistant Jurkat clones (ca. 3×10⁵/500 μl) were incubated for 3 hours at a temperature of 37° C. with 50 μl of the supernatant of neuro-2a-cells transfected with a FasL expression vector, or with a control supernatant of cells transfected with a sham vector (Rensing-Ehl, A. et al. Eur. J. Immunol. 25, 2253-2258 (1995)). The Jurkat cells are washed with FACS buffer (2% icFCS and 0.02% acid) and fixed in 70% ice cold ethanol. After washing for another time with FACS buffer treatment with RNAse for 5 minutes at a temperature of 37° C. followed (50 μg/ml RNAse A in 100 mM Tris HCl, pH 7.4, 100 mM NaCl, 5 mM EDTA). Staining for analysis of the DNA content proceeded with 250 μg/ml propidium iodide in PBS/1% NP40. Apoptotic cell fraction was analyzed and quantified in a Becton-Dickinsen FACScan-apparatus using the Lysis II software. The susceptibility of Raji clones to anti-APO1 induced apoptosis was analyzed by means of cell incubation (5×10⁵/ml) with varying concentrations of monoclonal anti-APO1 antibody (in medium for 16 hours at 37° C.). Quantification of the DNA fragmentation as a measure of the extent of the apoptosis in this case was mainly carried out as described by Nicoletti, I. et al., J. Immunol. Methods 139, 271-279 (1991). In sum it may be said that the cells were washed once with PBS and were carefully resuspended in. 0,1% sodium citrate and 0.1% triton x-100 with 50 g/ml propidium iodide. After incubation at 40 in darkness for a duration of at least 24 hours, the percentage of the apoptotic cell nuclei was determined by FACScan® (Becton-Dickinsen, Heidelberg, Germany).

For the quantification of the apoptosis with the transient transfected 293T cells, cells from a 5 cm plate were lysed in 200 μl incubation buffer, and lysates from 25,000 cells were analyzed for presence of histone DNA complexes by means of a Cell Death Detection ELISA (Boehringer Mannheim) according to manufacturer's specifications. The survival of clones transfected with E8 or with human FLIP, as well as control Jurkat clones after cell death induction after adding TRAIL was tested after a 20-hour incubation of ca. 50,000 cells per well at the indicated concentrations of recombinant TRAIL with a FLAG appended and 1 μg/ml of monoclonal antibody anti-Flag, and the proliferating cells were subsequently quantified with a cell titer AQ proliferation test (Promega), likewise according to manufacturer's specifications.

The viral in vitro cultures and the Northern blot analysis of transcripts were carried out as with Fickenscher et al., (J. Virol. 70, 6012-6019 (1996)). The effect of an HVS-infection on the cell death of owl monkey kidney cells (OMK) initiated through CD95L was tested by distributing the cells to 96 well plates at a concentration of ca. 10⁴ cells/well. Two days later half of the wells were infected with viruses to such an extent that the proportion of the infectious agent was about one virus per cell. Recombinant sCD95L (sFasL) (as described by Bodmer, J. L. et al., Immunity 6, 79-88 (1997)) was added at a concentration of 0.3 μg/ml after the infection at different points in time. The samples were examined 20 hours later for presence of histone-DNA complexes, as described above.

### Example 1

To test the inhibitory effect of the viral FLIP proteins on apoptosis and to show that proteins with death effector domains might also have an inhibitory effect on apoptosis, 239T cells with expression vectors for coding FLIP protein (from virus EHV-2) marked with Flag for FADD or N-terminal were used. The promoters for the expression vectors in each case were CMV vectors. After transfection of the cells cell extracts were examined by the so-called Western blot method. The proteins present in the cell lysates were separated in one direction according to their isoelectric points, and in the other direction of the two-dimensional probe were recorded according to size. Through the appropriate antibodies the expression of the desired proteins may then be examined. In the case at hand anti-Flag antibodies (attached to the N-terminal end of the FLIP protein) and anti-Fadd antibody were used to identify a stable transfection of the 293T cells. The corresponding illustrations are found in the lower part of FIG. 5. In addition 293T cells were also transfected with a myc-CD95 construct (this is an apoptosis receptor). All in all five different transfection clones were produced. The transfection pattern of the various clones is shown in the upper part of FIG. 5a. For instance, the fourth from the last column shows 293T cell transfectants which have been transfected with Flag-E8, FaDD and my-CD95. After testing for a stable transfection by means of the corresponding proteins the mutual association of the individual proteins was investigated by means of the Western blots and with the aid of c-immune precipitation experiments shown in the lower section of FIG. 5a. This is done by immunoprecipitation with anti-Flag E8 antibodies. In the Western blot recording the proteins FaDD or myc-CD95 are recognizable only if they have previously been precipitated with the anti-Flag antibody as a Coprecipitate. Thus the detection of FADD and/or myc-CD95 is possible only if at the time of the antibody attachment the Flag-E8 construct was likewise associated with FADD and/or myc-CD95. Therefore the two first experiments (column 1, 2) in FIG. 5a, in which there was no Flag-E8 transfection, serve as control experiments. The last three columns (in each case Flag-E8, i.e. containing viral FLIP protein) of FIG. 5a show the binding behavior of viral FLIP to either FADD and/or myc CD95. Because this involves a denaturized gel, no associations show up in the Western blots. Instead, the linkage of the two proteins to the viral FLIP protein in the cell extract is demonstrated indirectly by the presence of FADD and/or myc-CD95 after immune precipitation.

FIG. 5b shows an analogous experiment, in that here in a construct made up of Flag and the FLIP gene of the MCV virus (Open Reading Frame 159L) the 293 T-cells have been transfected. The experimental procedure is analogous to the procedure in FIG. 5a and described in detail in exemplary embodiment 7. For the immune precipitation see the method of Bodmer et al. (immunity 6, 79-88 (1997)).

In FIG. 5b as well the reveals that an association between the proteins myc-CD95 and Flag/-FLIP (159L) only exists if the 293T cells with all three expression vectors have been stably transfected. This may be seen in the right-hand column of FIG. 5b. On the other hand the two viral FLIP constructs (E8 or 159L) do not show any association with the CD95 receptor if the cells are not FADD-positive (FIG. 5a, third column, FIG. 5b, fourth column). In the reverse case the viral FLIP protein does not prevent the association of FADD with myc-cD95 (FIG. 5a, fourth column, FIG. 5b, fifth column), because after the co-immune precipitation in this case FADD and also myc-CD95 may be detected by means of the Western blot method.

### Example 2

The goal of the second example was an investigation of the incorporation of the FLIP protein (in this case the E8 FLIP protein) in the so-called DISC complex, which during activation of the apoptotic signal cascade is associated with the cytoplasmic portion of the CD95 receptor. The E8 FLIP gene served to transfect Raji clones in a stable fashion. Here expression vectors were also employed, with the promoter being an Srα promoter. The stably transfected Raji clones (RE8/11 and RE8/19) were compared with control clones which had only been transfected with the vector, but without insertion of the FLIP Gene. These are called RCo/1 and RCo/3. To identify a stable transfection, the expression of the transfected gene was investigated by means of Western blot analysis analogous to the first exemplary embodiment. In FIG. 6a in each case a band of the FLIP protein can be detected with the transfected clones RE8/11 and RE8/19, but clones RCo/1 and RCo/3, the control clones, do not exhibit any expression of the E8 protein. Here too a co-immune precipitation was undertaken, with anti-CD95 antibody. The co-immune precipitates then were separated by 2D gel electrophoresis under denaturization conditions (one of the axes records the SDS-PAGE and the other direction records the results by iso-electric focussing). An anti-Flag antibody, served to treat the blot and at the same time was directed against the viral FLIP protein.

FIG. 6b presents the result of this experiment. It is shown that only in the case Raji clone stably transfected with Flag E8 a positive signal for an approximately 23 kD protein with a pI value of about 5.0 could be observed. With the control clone without E8 expression (upper illustration in FIG. 6b) no corresponding signal can be detected. The E8 Flag construct gives rise to an expectation of a pI value of 5.0 and a molecular weight of approximately 23 kD.

### Example 3

This example seeks to demonstrate that the E8 FLIP protein has an effect on the constitutive structure of the DISC complex. DISC formation was therefore analyzed in detail. This was shown in a comparison involving the control clone RCo/3 and the clone RE8/19 which was stably transfected by means of Flag E8 FLIP (FIG. 7a). Anti-APO antibodies with ³⁵S-markers were used for this. The antibody has an agonistic effect on the death receptor Apo-1 (CD95). It was received from P. H. Krammer (DKFZ, Heidelberg, Germany). In the following the immune precipitate received using the above antibody was analyzed by 2D gel electrophoresis and the gel was evaluated by autoradiograph. The method used has by the way been described by Kischel et al. (EMBO Journal 14, 5579-5588 (1995)).

In addition the proteolytical activity in the disc complex against the FLICE protein was examined in Raji cells transfected with E8 and in control Raji cells. For this purpose the cells were either treated with anti-Apo-1 antibody for five minutes or they remained untreated. With the anti-Apo-1 antibody, immune precipitation was then carried out, and then the proteolytical activity of the immune precipitate from the anti-body treated and the untreated cells tested against FLICE. Proteolysis in vitro of the FLICE protein marked by ³⁵S was initiated by incubation with the immune precipitate. Subsequently the specific fission products of the FLICE protein (p43, p26, p17, p12 and p9) were examined by autoradiography on an SDA emulsion.

The result of the investigations with the Raji control clones and the Raji clones transfected with E8 shows characteristic differences. Above all, the typical DISC proteins CAP4 (═FLICE) and CAP3 (a FLICE derivative) are missing in the DISC complex of the Raji clones transfected with E8. This means that an orderly structure of the DISC complex as with the control cells, no longer exists while the E8 protein is present (FIG. 7a). Functionally there is another marked difference with regard to the fission activity of the FLICE protein, insofar as in the case of the RE8/19 clones treated with anti-Apo-1 antibody, in contrast to the control clones, the specific fission products of the FLICE protein can almost no longer be detected (FIG. 7b).

### Example 4

To further prove the significance of the viral FLIP protein for inhibiting apoptosis, cell death of several cells through various agonists was analyzed in the absence and presence of viral FLIP protein. For this purpose at first the number of apoptotic cells was measured in relation to the number of the anti-Apo-1 antibodies. Quantification of the apoptotic cells occurred as explained above. Here too the apoptotic cells of control clones RCo/1 and RCo/3 were analyzed in a comparison with the E8 FLIP transfected Raji B cell clones. The induction of the apoptotic signal cascade took place through agonistic anti-Apo-1 antibodies (FIG. 8a). In addition cell extracts of E8 transfectants (JE8/1, JE8/5, JE8/10, and JE8/13), and of control cells (Jco/2, Jco/3, Jco/4) Jurkat clones were analyzed. For this purpose the Flag-E8-FLIP expression was determined by means of anti-Flag antibody on Western blots. As described above, after an incubation of 3 hours at 37° C., and induced by CD95L, cell death was measured (with the aid of supernatant of neuronal cells). As described above in connection with FIG. 8, the cells marked with propidium iodide were examined by FacScan flow-through cytometer as to their apoptotic reaction.

The over-expression of CD95 receptor in human embryo kidney cells (293T cells) was determined as another apoptotic agent. Single transfectants of 293T cells were produced with an expression vector coding for CD95, as well as double transfectants with an expression vector coding for E8 or CD95. During over expression of CD95 (2 μg), massive cell death is observed. Hereby the relative quantity of DNA histone complexes liberated into the cytoplasm is measured. In a typical CD95 transfection experiment about 50-90% of cells succumb to apoptosis due to over-expression of the CD95 receptor. For comparison a CD95 single transfectant was also treated with the protease inhibitor z-VAD-fmk (25 μM).

The result of all three experiments shows that after stimulation of the apoptotic reaction, perhaps by means of an agonistic anti-Apo-1 antibody (FIG. 8a), by CD95L or over-expression of the CD95 receptor, the apoptotic reaction may be blocked or at least to a large extent reduced if the stimulated cells have previously been stably transfected with viral FLIP. In analogous experiments single and double transfectants of 293T-cells were transfected with expression vectors for TRAMP (Bodmer et al. Immunity 6, 79-88, (1997), each time with different quantities of expression vectors either for E8-FLIP (EHV-2) or 159L-FLIP (MCV) or 71-FLIP (HVS). As controls false transfectants (mock) without TRAMP or FLIP protein expression were chosen. Incremental amounts of expression vectors were used with the FLIP proteins, while the amounts of TRAMO expression vectors was kept constant (3 μg) in all experiments. The result (summarized in FIG. 9) shows that increasing amounts of viral FLIP protein expression vectors can markedly reduce the amount of apoptosis caused by over-expression of TRAMP receptors.

### Example 5

This example served to investigate when viral FLIP protein is expressed during the viral replication cycle. The HVS-71-FLIP protein was chosen for this purpose. OMK cells (Owl Monkey Kidney) were used as host cells for the viral infection. Northern blot analysis was used to analyze the transcription of permissive OMK cells with an HVS infection of the C₄₈₈ strain. An mRNA determination of the viral FLIP gene in the cells infected with the HVS virus was undertaken one, two, three or four days after infection. Non-virus-infected OMK cells were examined as controls. Two additional mRNA analyses were performed, on the one hand with HVS-infected T-cells of the line P-1079 and on the other with OMK cells that were infected with the HVS strain All. With the last-mentioned cell line the samples were examined by Northern blot test four days after infection with the virus strain.

FIG. 11a shows the result that the permissive OMK cells that had been infected with a cytopathological HVS strain (C488 or A11), on the fourth day after infection showed a numerous presence of a 5 kb mRNA fragment. This mRNA fragment serves to translate the viral FLIP protein. Therefore the FLIP transcript appears one day before massive cellular lysis. This may be seen in FIG. 11b.

### Example 6

To determine the binding of human FLIP_{S} and FLIP_{L} to CD95 via the adapter molecule FADD, cotransfection experiments were conducted in human embryo kidney cells (293 cells), which constitutively express SV40 large T antigen and therefore exhibit a more vigorous protein expression of expression vectors with an SV40 (293T cells).

The expression vector pCR-3 of Invitrogen has this property. Therefore DNA fragments coding for human proteins or protein segments FLIP_{S}, FLIP_{L} and FLIP_{P} were cloned for expression in 293T cells in a modified version of the vector pCR-3, which provides these proteins or protein segments with an N-terminal Flag-epitope, while the DNA site coding for the cytoplasmic protein segment of CD95 was inserted in an analogous expression vector with an N-terminal myc epitope. As shown in FIG. 12, various combinations of expression vectors were transfected for the cytoplasmic part of CD95, for FADD, FLIP_{S}, FLIP_{L} and FLIP_{P} transfected in 293T, and the expression of the gene products coded by the various expression vectors was controlled by specific anti-Flag, anti-FADD, or anti-myc antibody in the Western blot. In addition the Flag-marked FLIP_{S}, FLIP_{L} and FLIP_{P} present in cell lysates of appropriately transfected 293T cells, were, as described above, immune-precipitated by means of anti-Flag agarose, and these immune precipitates were then analyzed for association of FADD or the myc-marked CD95 protein segment in the anti-FADD or anti-myc Western blot.

Result

It was determined that the human proteins FLIP_{S} and FLIP_{L}, but not a protein segment of the human FLIP_{L}, which only includes the caspase-homologous inactive protease domain (FLIP_{P}), bind to the adapter molecule FADD. This binding does not inhibit the attachment of FADD to the cytoplasmic protein segment of the CD95 death receptor and as a result the human proteins FLIP_{S} and FLIP_{L} can bind to the cytoplasmic part of the death receptor CD95.

### Example 7

To establish that human FLIP_{S} and FLIP_{L} bind to FLICE, co-transfection experiments were undertaken in 293T cells described above (see FIG. 13). The expression vector for human FLICE (a gift from M. Peter, Heidelberg) provides this protein with an N-terminal HA epitope, while the expression vectors for FLIP_{S}, FLIP_{L} and FLIP_{P} as described above, provide these proteins or protein segments with an N-terminal Flag epitope. The expression of the gene products coded by the appropriate expression vectors in Western blot with specific anti-Flag or anti HA antibodies. The expression of the gene products coded by the various expression vectors was controlled by specific with specific anti-Flag or anti-HA antibodies in the Western blot. The Flag-marked FLIP_{S}, FLIP_{L} and FLIP_{P} present in cell lysates of appropriately transfected 293T cells, were immune-precipitated by means of anti-Flag agarose, and these immune precipitates were then analyzed in the anti-HA Western blot for association of HA-FLICE with FLIP_{S}, FLIP_{L} and FLIP_{P}.

Result

It was determined that the human proteins FLIP_{S} and FLIP_{L} bind to the caspase FLICE. Both the N-terminal protein segment with the two death effector domains in FLIP_{S} and the C-terminal protein segment FLIP_{P}, including the caspase homologous inactive protease domain, contribute to the binding of FLIP_{L} to FLICE.

### Example 8

To establish the inhibiting effect of FLIP_{S} and FLIP_{L} on the apoptosis induced by the death receptor CD95, a human Jurkat T-cell line and a human Raji B-cell line were transfected with an expression vector for human FLIP_{S} and FLIP_{L} provided with an N-terminal VSV epitope. An expression vector with an Srα promoter lending puromycin resistance to the transfected cells was used for stable transfection of these cells (The Vector was a gift from R. Sekaly, ICRM, Montreal, Canada). The cells were transfected by electroporation of 8×10⁶ cells at 250V and 960 μF in HeBS buffer solution with 20 μg of the plasmid to be transfected. After growing in culture medium without puromycin as described above, they were seeded in flat bottom cell culture plates with 96 wells after selection in culture medium with 5 μg/ml puromycin at 2000-20,000 cells each well. Within 2-3 weeks Puromycin resistant clones grew up and were then tested for expression of VSV-marked FLIP_{S} or FLIP_{L} in Western blot. Clones with different expression levels of FLIP_{S} or FLIP_{L} were then tested for their resistance toward apoptosis induced by sFasL (see FIG. 14). To achieve this the clones were incubated in culture medium with Flag-marked sFAsL and 1 μg/ml cross-linked anti-Flag antibody in the concentrations indicated in FIG. 14 for 20 h and at 37° C., and then the cell viability of the cells thus treated was determined through a cell proliferation assay (cell titer 96 AQ, Promega).

Result

It was determined that the human T-cell line Jurkat and the human B-Cell line Raji acquire a resistance to the apoptosis induced by the CD95 death receptor by expression of the human FLIP_{S} or FLIP_{L}. The expression of the longer form of the human FLIP (FLIP_{L}) offers more efficient protection against the apoptosis induced through CD95 that the shorter form of the human FLIP (FLIP_{S}), which does not include the caspase-homologous inactive protease domain.

### Example 9

To establish the inhibiting effect of FLIP_{S} and FLIP_{L} on the apoptosis induced by the death receptor ligand TRAIL, a human Jurkat T-cell line was, as described in the above example embodiment, transfected with the expression vector for human FLIP_{S} and FLIP_{L} provided with an N-terminal VSV epitope and clones raised under puromycin selection, which then were tested for VSV-marked human FLIP_{S} or FLIP_{L} in Western blot assays (see upper part of FIG. 14a). Clones with different expression levels of FLIP_{S} or FLIP_{L} were then tested for their resistance to apoptosis induced by TRAIL (see FIG. 15). To achieve this, the clones were incubated in culture medium for 20 hours and at 37° C. with Flag-marked TRAIL and 1 μg/ml cross-linked anti-Flag antibody in the concentrations indicated in FIG. 15, and then the cell viability of the cells thus treated was determined through a cell proliferation assay (cell titer 96 AQ, Promega).

Result

It was determined that the human T-cell line Jurkat acquires a resistance to apoptosis induced by the death receptor ligand TRAIL through expression of the human FLIPS or FLIP_{L}. The expression of the longer form of the human FLIP (FLIP_{L}) offers more efficient protection against apoptosis induced through CD95 than the shorter form of the human FLIP (FLIP_{S}), which does not include the caspase-homologous inactive protease domain.

### Example 10

Tissue homeostasis is maintained through an even balance between cell growth and apoptosis. While apoptotic signal transduction is responsible for the cell death of superfluous or infected cells, cell growth balances out certain cell losses. With numerous infectious diseases or with malignant tumors, this balance has been upset. Tumor diseases are characterized by either a site-specific or site-diffuse, accelerated proliferation of cells. In tumor cells, regulation of cell division has been lost. In tumor cells an effective apoptosis no longer takes place. Thus there are clear experimental indications that tumor cells, such as melanomas or hepatomas, do not react with cell death to binding with CD95L. This is possible through “down-regulation” of the CD95 expression or a blockage within the signal transduction path (Hahne, M. et al., Science 274, 1363 (1996) Strand, S. et al., Nature Med 2, 1361-1366 (1996)).

In fact, further experiments have shown that FLIP expression in melanocytic tumors (malignant melanomas) increases with malignant progression. That is, the percentage of FLIP positive cells in advanced lesions (metastases) is significantly higher than in less advanced lesions (vertical growth phase melanoma and superficial spreading melanoma). This data provides evidence showing that tumor cells are progressively selected in vivo for elevated FLIP expression, most likely due to selective pressure by the immune system.

Experiments have also show that transfection of melanoma cell lines with FLIP expression vectors renders these cells more resistant to FasL and TRAIL than mock-transfected controls. This provides direct evidence that FLIP inhibits death receptor-mediated cell death in melanomas, and that the level of cellular FLIP expression correlates inversely with cellular sensitivity to death ligands such as FasL and TRAIL. Thus, inhibition of death receptor signaling (Fas down regulation by Ras for example) in Fas-expressing cells significantly favors tumor development. This creates a certain state of immune-tolerance to the tumor.

Moreover, expression of death ligands (including FasL) by tumors favors tumor outgrowth by killing immune cells (including cytotoxic lymphocytes (CTL's) and antigen presenting cells) implicated in the immune response against the tumor. This also creates a certain state of immune-tolerance to the tumor.

### Applications

The effect of viral FLIP proteins is based on an inhibition of the apoptotic signal transduction mechanism as described above. Viruses have integrated the presumably originally cellular genetic material with their genome in order to escape the virus-specific immune response of the immune system. Due to the presence of the viral FLIP proteins as an inhibitor of apoptosis the immune reaction is incapable of killing off the virus-infected immune cell and thereby to interrupt the reproductive cycle of the virus. The integration of the FLIP protein into the viral genome thus promotes viral spread, i.e., the consistent infection of the host.

The integration of the virus or the expression of the FLIP protein may, however, also have a transformative effect. In this connection it is interesting that of all things, numerous herpes viruses have transformational properties, i.e., properties that allow the normally regulated cell to become a tumor cell. The identification of the inhibiting viral FLIP proteins according to the invention makes a decisive contribution to understanding the connection between transformations and the origin of tumors, which hereby is being disclosed. Viral FLIP protein also plays a key role in understanding viral tumor origination. Numerous data confirm the correlation between virus persistence and the origination of tumors. Thus virus MCV produces slow-growing epidermal neoplasms, which may escape immune resistance for a long time. The virus HVS causes tumors in certain primate groups, and there are indications through dermatological studies that the virus HHV-8 also operates as an infectious co-factor for the Kaposi sarcoma, which particularly occurs with AIDs patients, as well as for certain forms of primary lymphoma with regard to the transformation process.

Despite evidence for the in vivo generation of tumor specific CTL's, spontaneous regression of cancer only rarely occurs. The mechanisms thought to be responsible for this tumor immune escape to date include the expression of local inhibitory factors by tumor cells, such as transforming growth factor (TGF) β, IL-10 and FasL; deficient antigen processing by tumor cells or loss of MHC expression; the lack of immunogenicity and costimulation for CTL activation, and defective lymphocyte homing to the tumor. Overexpression of FLIP can ow be added to this list.

Current attempts to improve cancer survival depend essentially on early diagnosis, and the development of new treatment modalities, on of the most promising being immunotherapy. Given the new findings described herein, strategies to modulate FLIP expression and/or FLIP-mediated inhibition of death receptor signaling should prove to be a useful complementary approach to the treatment of cancer, and also to the prevention of graft rejection (cell or organ).

With the above in mind, the following strategies to modulate FLIP expression and or function will be useful for cancer treatment and therapies to prevent graft cell or organ rejection.

1) Strategies to block the function of FLIP (e.g., its interaction with caspases) will sensitize cells to death ligands (e.g., FasL and TRAIL).

2) Strategies to block the function of FLIP in tumor cells will significantly inhibit tumor development and/or growth.

3) Strategies to block the function of FLIP in tumor cells will have potential for the treatment of cancer.

4) Strategies to block the function of FLIP in tumor cells will favor tumor clearance by the immune system, including tumor specific CTL's. These strategies will therefore also enhance the efficacy of cancer immunotherapies including cell based, antigen-specific, and dendritic cell vaccines, as well as biological response modifiers such as DC40L, GM-CSF, and RANk-L.

5) Strategies to block the function of FLIP in tumor cells will partially or completely lift the state of tolerance of their immune systems toward tumors.

6) Strategies to enhance the expression and/or function of FLIP in immune cells (including cytotoxic lymphocytes (CTL's) and antigen presenting cells) implicated in the immune resnonse against the tumor, will enhance anti-tumor immune function and the efficacy of cancer immunotherapies (including cell based, antigen-specific and dendritic cell vaccines as well as biological response modifies such as CD40L, GM-CSF, and RANk-L.

7) Strategies to enhance the expression and/or function of FLIP in grafted cells (including semi-allogeneic, allogeneic, and xenogeneic) or organs, will inhibit death receptor-mediated graft destruction/failure, and thus improve graft tolerance/survival.

Furthermore, all γ-herpes viruses that code for a FLIP protein also have a bcl-2 homologue. The anti-apoptotic bdl-2, however, blocks cell death which is initiated by external influences, such as deprivation of growth factors, γ-radiation or cytotoxic substances. In contrast to the gene products of the viral DNA sequences in accordance with the invention, their anti-apoptotic effect with lymphocyte cell lines through stimulation of the CD95 receptor is less developed. Therefore, the above-mentioned viruses have two genotypes with complementary properties, for instance, one bcl-2 homologue and one vFLIP protein. According to the invention, by combining these two genotypes, for instance two different expression vectors carrying these genotypes, an anti-apoptotic property toward many environmental influences may be achieved for the cell. According to the invention, making a recombinant protein with effective domains of bcl-2 homologues and effective domains of vFLIP proteins available is also conceivable.

While the genes or gene products according to the invention make a considerable contribution to the immortalizing process of tumor cells and thus function as tumor agents, on the other hand, with the aid of the sequences according to the invention, or their gene products according to the invention, an immortalizing process can be initiated if so desired. This is true in particular for illnesses which, due to an unregulated stimulation of the apoptosis mechanism, lead to frequent or massive cell loss. In this connection auto-immune diseases must be mentioned (for instance, rheumatoid arthritis or lupus erythematosus, or multiple sclerosis). The appropriate application of the DNA sequences according to the invention, of their gene products according to the invention, the expression vectors derived from these, or host cells transformed with these expression vectors could prevent such cell death.

In particular an immortalization of the cells affected by autoimmune disease must also be considered. In the form of gene-therapeutic procedures these cells could be transfected with the DNA sequences according to the invention, for instance also in vitro, and the cells could then be re-transplanted.

In the case of HIV infections an application of the subjects of the invention is another possibility. FLIP proteins with their potential for inhibiting the apoptotic signal transduction mechanism could then be integrated into cells, preferably T-cells, ex vivo, and after retransplantation of the cells, after extra-cellular stimulation of the cells in vivo, be kept from dying off. These cells could survive in the patient without limit and exercise their immunological functions. Thus, in case of HIV infections, immune cells might be saved from mass destruction.

Laboratory application is another possibility. Lab medicine and/or the biomedical field has for decades been hampered by the problem that certain cell lines do not survive several generations of laboratory cultivation. The limited cell division of certain cell lines does not permit any thorough examination of their cellular or physiological properties in vitro. By means of transfection of cell lines with the DNA sequences according to the invention, immortality may be achieved for these cell lines.

### Other Embodiments

Other embodiments are within the following claims.

1. An antisense nucleic acid that inhibits expression of a FLIP protein encoded by the nucleotide sequence of SEQ ID NO:14 or SEQ ID NO:16.

2. The antisense nucleic acid of claim 1, wherein the nucleic acid is an oligonucleotide.

3. The antisense nucleic acid of claim 2, wherein the oligonucleotide is 20 to 40 nucleotides in length.

4. The antisense nucleic acid of claim 1, wherein the nucleic acid is DNA.

5. The antisense nucleic acid of claim 1, wherein the nucleic acid is RNA.

6. The antisense nucleic acid of claim 1, wherein the nucleotide sequence is SEQ ID NO:14.

7. The antisense nucleic acid of claim 1, wherein the nucleotide sequence is SEQ ID NO 16.

8. An expression vector comprising the antisense nucleic acid of claim 1.

9. A host cell comprising the expression vector of claim 8.

10. The host cell of claim 9, wherein the host cell is a eukaryotic cell.

11. The host cell of claim 9, wherein the host cell is a human cell.

12. A method of decreasing FLIP expression in a cell, the method comprising contacting a cell with an amount of the antisense nucleic acid of claim 1 effective to decrease FLIP expression in the cell.

13. A method of decreasing FLIP expression in a cell, the method comprising contacting a cell with an amount of the antisense nucleic acid of claim 2 effective to decrease FLIP expression in the cell.

14. A method of decreasing FLIP expression in a cell, the method comprising contacting a cell with an amount of the expression vector of claim 8 effective to decrease FLIP expression in the cell.

15. A method of inducing apoptosis in a cell, the method comprising contacting a cell with an amount of the antisense nucleic acid of claim 1 effective to induce apoptosis in the cell.

16. A method of inducing apoptosis in a cell, the method comprising contacting a cell with an amount of the antisense nucleic acid of claim 2 effective to induce apoptosis in the cell.

17. A method of inducing apoptosis in a cell, the method comprising contacting a cell with an amount of the expression vector of claim 8 effective to induce apoptosis in the cell.


SEQUENCE LISTING (27 sequences)

SEQ ID NO: 1 - protein, 171 aa, Equines herpesvirus 2
MSHYSMIDTYFSLDEDETETYLYLCRDLLKNKGEFQCTRDAFKFLSDYACLSAANQMELLFRVGRLDLIRRIFGQTWTPDSCPRYYMPICSPFRCLMALVNDFLSDKEVEEMYFLCAPRLESHLEPGSKKSFLRLASLLEDLELLGGDKLTFLRHLLTTIGRADLVKNLQV

SEQ ID NO: 2 - protein, 169 aa, Humanes herpesvirus 8
MATYEVLCEVARKLGTDDREVVLFLLNVFIPQPTLAQLIGALRALKEEGRLTFPLLAECLFRAGRRDLLRDLLHLDPRFLERHLAGTMSYFSPYQLTVLHVDGELCARDIRSLIFLSKDTIGSRSTPQTFLHWVYCMENLDLLGPTDVDALMSMLRSLSRVDLQRQVQT

SEQ ID NO: 3 - protein, 167 aa, Saimirine herpesvirus 2
MDLKTTVLHITDSFTDEEMYCLLFLINGCIPRSCNAVNISDLIIETLSKSTQWDICLMQCLYVLRKIGLLLNLFQVTKEAVKQSFFTQPQLETHVLTLVNVNNNLTAKDEKRLCFILDQFFPRNVAAPSVILCVFSNMLCEMHVLECLCQLKKCLKQIGRSDLAKTV

SEQ ID NO: 4 - protein, 171 aa, Bovines herpesvirus 4
MVTRDVLLAIETHLNQNEKTFVMYFLLDPYIPKECKDFLPTLENLHSKRKIRYPILIELMYILQRFDLLRSIFLLDHRFVKDQITSSHWKYISPYKQLIFSIGQNIDDEDLISIKFISMNYIGKSPSKIKNYLDWVRALEKVDMVGPDNLDLFETFFKQIHRMDIVKMIKN

SEQ ID NO: 5 - protein, 169 aa, Molluscum contagiosum virus subtype 1
VPSLPFLRHLLEELDSHEDSLLLFLCHDAAPGCTTVTQALCSLSQQRKLTLAALVEMLYVLQRMDLLKSRFGLSKEGAEQLLGTSFLTRYRKLMVCVGEELDSSELRALRLFACNLNPSLSTALSESSRFVELVLALENVGLVSPSSVSVLADMLRTLRRLDLCQQLVE

SEQ ID NO: 6 - protein, 165 aa, Molluscum contagiosum virus subtype 1
PIPFSFLRNLLAELDASEHEVLRFLCRDVAPASKTAEDALRALQRRRLLTLSSMAELLCALRRFDVLKVRFGMTRECAGRLLGHGFLSQYRLQVAAINNMVGSEDLRVMCLCAGKLLPPSCTPRCLVDLVSALEDAGAISPQDVSVLVTLLHAVCRYDLSVALSA

SEQ ID NO: 7 - protein, 177 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIIND

SEQ ID NO: 8 - protein, 170 aa, Homo sapiens
VSFREKLLIIDSNLGVQDVENLKFLCIGLVPNKKLEKSSSASDVFEHLLAEDLLSEEDPFFLAELLYIIRQKKLLQHLNCTKEEVERLLPTRQRVSLFRNLLYELSEGIDSENLKDMIFLLKDSLPKTEMTSLSFLAFLEKQGKIDEDNLTCLEDLCKTVVPKLLRNIEK

SEQ ID NO: 9 - protein, 81 aa, Homo sapiens
MDPFLVLLHSVSSSLSSSELTELKFLCLGRVGKRKLERVQSGLDLFSMLLEQNDLEPGHTELLRELLASLRRHDLLRRVDD

SEQ ID NO: 10 - protein, 81 aa, Homo sapiens
MVEYGTLFQDLTNNITLEDLEQLKSACKEDIPSEKSEEITTGSAWFSFLESHNKLDKDNLSIIEHIFEISRRPDLLTMVVD

SEQ ID NO: 11 - protein, 480 aa, Homo sapiens
MSAEVIHQVEEALDTDEKEMLLFLCRDVAIDVVPPNVRDLLDILRERGKLSVGDLAELLYRVRRFDLLKRILKMDRKAVETHLLRNPHLVSDYRVLMAEIGEDLDKSDVSSLIFLMKDYMGRGKISKEKSFLDLVVELEKLNLVAPDQLDLLEKCLKNIHRIDLKTKIQKYKQSVQGAGTSYRNVLQAAIQKSLKDPSNNFRLHNGRSKEQRLKEQLGAQQEPVKKSIQESEAFLPQSIPEERYKMKSKPLGICLIIDCIGNETELLRDTFTSLGYEVQKFLHLSMHGISQILGQFACMPEHRDYDSFVCVLVSRGGSQSVYGVDQTHSGLPLHHIRRMFMGDSCPYLAGKPKMFFIQNYVVSEGQLEDSSLLEVDGPAMKNVEFKAQKRGLCTVHREADFFWSLCTADMSLLEQSHSSPSLYLQCLSQKLRQERKRPLLDLHIELNGYMYDWNSRVSAKEKYYVWLQHTLRKKLILSYT

SEQ ID NO: 12 - protein, 481 aa, Mus musculus
MAQSPVSAEVIHQVEECLDEDEKEMMLFLCRDVTENLAAPNVRDLLDSLSERGQLSFATLAELLYRVRRFDLLKRILKTDKATVEDHLRRNPHLVSDYRVLLMEIGESLDQNDVSSLVFLTRDYTGRGKIAKDKSFLDLVIELEKLNLIASDQLNLLEKCLKNIHRIDLNTKIQKYTQSSQGARSNMNTLQASLPKLSIKYNSRLQNGRSKEPRFVEYRDSQRTLVKTSIQESGAFLPPHIREETYRMQSKPLGICLIIDCIGNDTKYLQETFTSLGYHIQLFLFPKSHDITQIVRRYASMAQHQDYDSFACVLVSLGGSQSMMGRDQVHSGFSLDHVKNMFTGDTCPSLRGKPKLFFIQNYESLGSQLEDSSLEVDGPSIKNVDSKPLQPRHCTTHPEADIFWSLCTADVSHLEKPSSSSSVYLQKLSQQLKQGRRRPLVDLHVELMDKVYAWNSGVSSKEKYSLSLQHTLRKKLILAPT

SEQ ID NO: 13 - protein, 171 aa, Equine herpesvirus 2; identical to SEQ ID NO: 1

SEQ ID NO: 14 - DNA, 1190 bp, Homo sapiens
  CDS (394)...(1056)
aagctcgaaattaaccctcactaaagggaacaaaagctggagctccaccgcggtggcggccgctctagaactagtggatcccccgggctgcaggaattcggcacgagcggcacgagtagacttctatagatccctttctatagaacttaatctacttaagtcagggagaccacccagaaggaaagagcccatactttcaatcttaggcataagttagcttgataagattttcagaaaaattcccttttaaccacagaactcccccactggaaaggattctgaaagaaatgaagtcagccctcagaaatgaagttgactgcctgctggctttctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggatgataacaccctatgcccattgtcctgatctgaaaattcttggaaattgttccatgtgattaacatggaactgcctctacttaatcattctgaatgattaaatcgtttcattttctaaatgtgtaaaaaaaaaaaaaaaaaaaaaaactcgagggggggcccgtacccaattcgccctatagtgagtcgt

SEQ ID NO: 15 - protein, 221 aa, Homo sapiens
MSAEVIHQVEEALDTDEKEMLLFLCRDVAIDVVPPNVRDLLDILRERGKLSVGDLAELLYRVRRFDLLKRILKMDRKAVETHLLRNPHLVSDYRVLMAEIGEDLDKSDVSSLIFLMKDYMGRGKISKEKSFLDLVVELEKLNLVAPDQLDLLEKCLKNIHRIDLKTKIQKYKQSVQGAGTSYRNVLQAAIQKSLKDPSNNFRMITPYAHCPDLKILGNCSM

SEQ ID NO: 16 - DNA, 2143 bp, Homo sapiens
  CDS (383)...(1822)
taggggtggggactcggcctcacacagtgagtgccggctattggacttttgtccagtgacagctgagacaacaaggaccacgggaggaggtgtaggagagaagcgccgcgaacagcgatcgcccagcaccaagtccgcttccaggctttcggtttctttgcctccatcttgggtgcgccttcccggcgtctaggggagcgaaggctgaggtggcagcggcaggagagtccggccgcgacaggacgaactcccccactggaaaggattctgaaagaaatgaagtcagccctcagaaatgaagttgactgcctgctggctttctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatacaaaaattagctgggtgtgggtgtgggtacctgtattcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaa

SEQ ID NO: 17 - protein, 480 aa, Homo sapiens; identical to SEQ ID NO: 11

SEQ ID NO: 18 - DNA, 2452 bp, Mus musculus
  CDS (172)...(1614)
gaattccgagcctctcaagcggccacttagggccggacagagtgtctctattgcaagaactctgagagaaatgaagagagtcctcagcaatgatgttggcttctcgtggttcccagagccctgcttaatggatggagactggacgagaacctggctgctgtggttctgaacatggcccagagccctgtgtctgccgaggtcattcaccaggtggaagagtgtcttgatgaagacgagaaggagatgatgctcttcctgtgtagagatgtgactgagaacctggctgcacctaacgtcagggacctcctggatagcttaagtgagagaggccagctctcttttgctaccttggctgaattgctctacagagtgaggcggtttgaccttctcaagaggatcttgaagacagacaaagcaaccgtggaggaccacctgcgcagaaaccctcacctggtttctgattatagggtcctgctgatggagattggtgagagcttagatcagaacgatgtatcctccttagttttccttacaagggattacacaggcagaggcaagatagccaaggacaagagtttcttggatctggtgattgaattggagaaactgaatctaattgcttcagaccaattgaatttgttagaaaaatgcctgaagaacatccacagaatagacttgaacacaaagatccagaagtacacccagtccagccaaggagcaagatcaaatatgaatactctccaggcttcgctcccaaaattgagtatcaagtataactcaaggctccagaatgggcgaagtaaagagccaagatttgtggaataccgtgacagtcaaagaacactggtgaagacatccatccaggaatcaggagcttttttacctccgcacatccgtgaagagacttacaggatgcagagcaagcccctaggaatctgcttgatcattgattgtattggcaacgacacaaaatatcttcaagagaccttcacttccctgggctatcatatccagcttttcttgtttcccaagtcacatgacataacccagattgttcgccgatatgcaagtatggcccaacatcaagactatgacagctttgcatgtgttctggtgagcctaggaggctcccaaagcatgatgggcagagatcaagttcactcagggttctccttggatcatgtcaagaacatgttcacgggggacacgtgcccttctctcagagggaagccaaagctcttttttattcagaactatgagtcgttaggtagccagttggaagatagcagcctggaggtagatgggccatcaataaaaaatgtggactctaagcccctgcaacccagacactgcacaactcacccagaagctgatatcttttggagcctgtgcacagcagacgtatctcacttggagaagccctccagctcatcctctgtgtatctgcagaagctctcccagcagctgaagcaaggcaggagacgcccactcgtggacctccacgttgaactcatggacaaagtgtatgcgtggaacagtggtgtttcgtctaaggagaaatacagcctcagcctgcagcacactctgaggaagaaactcatcctggctcctacgtgagaaccccagaccgttggtgttcttggtatatcatccagggtggcggcttggagcagagcttggcggttacggctgcttctggctgcttctggctctgccgtgagtcctggcctagggttctcctgtgcacaggcatgagccgtaaccctgtgcctgggaaacgtctcactcccgccgccgtgcctttacctctctaaacttccctacttacattccttagtcggatgttttgccagagtgtggagaacagtaagacataaacctattgtttgtttgtttttttgggggggaggttatctaccaagttataccaagttattgtatgggtgtatagtgtatagtggttcaagatttgacactgaatgtaacttgagacttacctgagtttgtcatgcgactgggtaaattgtttctatggcacatctaatcatttaataagtaattacctcattaagtacccattgcttcaggactttcacattggccaccaatttctgtgacccagctccacatttatattctctttctgcaaaaccaaatttcattatgtctgtttaatatctacagtctaatgctttgtaagacatctagatagaaaaatagttacccatgagcacagaagggctggcctgaccctcaccagctgtgcagtggcttcggtgaaggagaatgagccctactccttgaaggttgtagtgcttgggagagcagtctgtaccttgcctgggcagcacagtagagccagccccaagaacacaacagtgagtgggggagcttgccctggttggctcaggatcaggaacaggagggatgacggaattcctgcagcccgggg

SEQ ID NO: 19 - protein, 481 aa, Mus musculus; identical to SEQ ID NO: 12

SEQ ID NO: 20 - protein, 479 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 21 - protein, 571 aa, Homo sapiens
MKSQGQHWYSSSDKNCKVSFREKLLIIDSNLGVQDVENLKFLCIGLVPNKKLEKSSSASDVFEHLLAEDLLSEEDPFFLAELLYIIRQKKLLQHLNCTKEEVERLLPTRQRVSLFRNLLYELSEGIDSENLKDMIFLLKDSLPKTEMTSLSFLAFLEKQGKIDEDNLTCLEDLCKTVVPKLLRNIEKYKREKAIQIVTPPVDKEAESYQGEEELVSQTDVKTFLEALPQESWQNKHAGSNGNRATNGAPSLVSRGMQGASANTLNSETSTKRAAVYRMNRNHRGLCVIVNNHSFTSLKDRQGTHKDAEILSHVFQWLGFTVHIHNNVTKVEMEMVLQKQKCNPAHADGDCFVFCILTHGRFGAVYSSDEALIPIREIMSHFTALQCPRLAEKPKLFFIQACQGEEIQPSVSIEADALNPEQAPTSLQDSIPAEADFLLGLATVPGYVSFRHVEEGSWYIQSLCNHLKKLVPRMLKFLEKTMEIRGRKRTVWGAKQISATSLPTAISAQTPRPPMRRWSSVSHEDILSILTAVNDDVSRRVDKQGTKKQMPQPAFTLRKKLVFPVPLDALSI

SEQ ID NO: 22 - protein, 221 aa, Homo sapiens; identical to SEQ ID NO: 15

SEQ ID NO: 23 - protein, 188 aa, Human herpesvirus 8
MATYEVLCEVARKLGTDDREVVLFLLNVFIPQPTLAQLIGALRALKEEGRLTFPLLAECLFRAGRRDLLRDLLHLDPRFLERHLAGTMSYFSPYQLTVLHVDGELCARDIRSLIFLSKDTIGSRSTPQTFLHWVYCMENLDLLGPTDVDALMSMLRSLSRVDLQRQVQTLMGLHLSGPSHSQHYRHTP

SEQ ID NO: 24 - protein, 182 aa, Bovine herpesvirus 4
MVTRDVLLAIETHLNQNEKTFVMYFLLDPYIPKECKDFLPTLENLHSKRKIRYPILIELMYILQRFDLLRSIFLLDHRFVKDQITSSHWKYISPYKQLIFSIGQNIDDEDLISIKFISMNYIGKSPSKIKNYLDWVRALEKVDMVGPDNLDLFETFFKQIHRMDIVKMIKNYRTRETLQITL

SEQ ID NO: 25 - DNA, 24 bp, Artificial Sequence
  oligonucleotide for PCR
tcagctgcagacctctgaggaaac

SEQ ID NO: 26 - DNA, 20 bp, Artificial Sequence
  oligonucleotide for PCR
actagttagccaactaaaaa

SEQ ID NO: 27 - protein, 8 aa, Artificial Sequence
  connecting element
GPGQVQLQ